(19) United States
(12) Patent Application Publication
(10) Pub. No.: US 2026/0041710 A1
(43) Pub. Date: Feb. 12, 2026

(54) A NOVEL COMBINATION FOR ITS UTILITY IN MICRO AND MACRO VASCULAR COMPLICATIONS

(71) Applicants: Yogesh BENDALE, Pune (IN); Vineeta BENDALE, Pune (IN)

(72) Inventors: Yogesh BENDALE, Pune (IN); Vineeta BENDALE, Pune (IN)

(21) Appl. No.: 18/852,239

(22) PCT Filed: Mar. 30, 2023
(86) PCT No.: PCT/IN2023/050314

§ 371 Date: Sep. 27, 2024

(30) Foreign Application Priority Data
Mar. 30, 2022 (IN) 202121044468

(51) Int. Cl. A61K 33/242; A61K 9/00; A61K 33/30; A61K 36/185; A61K 36/9066; A61P 27/02

(52) U.S. Cl. CPC A61K 33/242; A61K 9/0053; A61K 33/30; A61K 36/185; A61K 36/9066; A61P 27/02

(57) ABSTRACT

The present invention discloses a novel orally administered combination of bio-gold and bio-zinc, a process for its synthesis, a pharmaceutical composition comprising the combination and its applications, thereof in micro and macro vascular complications.

### FIELD OF THE INVENTION

The invention relates to the field of Pharmaceuticals. In particular, the present invention, discloses a novel orally administered combination of bio-gold and bio-zinc, a process for its synthesis, a pharmaceutical composition comprising the combination and its applications, thereof in micro and macro vascular complications.

### BACKGROUND OF THE INVENTION

The macro and microvasculature plays an important role in functioning of various vital organs. However, these vasculatures are at risk of damage due to various systemic disorders which either effect the biochemical composition of blood or the pressure exerted on the blood vessels. The microvasculature is made up of large elastic arteries. This elasticity is very important as it buffers the increase in pulsatility that occurs because of the effect of intermittent left ventricular contraction and more muscular arteries that act as a conduit to deliver a steady flow of blood to the microvasculature.

The microvasculature is defined as vessels with a diameter <150 μm in diameter and includes arterioles, capillaries, and venules. In the presence of systemic illness like Diabetes mellitus and hypertension, the large elastic arteries loose elasticity and thus have reduced buffering capacity. This may increase the adverse effect of the transmission of pulsatility from the macrocirculation to the macrocirculation, thereby having adverse effect on the vasculature. This may in turn increase the propensity to develop adverse structural and functional changes in many organs, including the heart, arterial system, brain, eye, and kidney (termed target organ damage).

Diabetes, one of the major complications of such macro and microvascular disorder, has been studied extensively in diabetic animals and humans with respect to the endothelial dysfunction which is an important cardiovascular risk factor owing to its a critical and initiating nature in the genesis of vascular diseases. Atherosclerotic lesions (both morphologically and functionally) give an indication of diabetic macrovascular disease. Diabetic microvascular disease is observed in patients in the form of retinopathy, neuropathy, nephropathy, and vascular abnormalities in the lower extremities. These illnesses may further lead to kidney failure, diabetic cardiomyopathy, stroke, and lower extremity dysfunction and visual impairment, etc. Chronic hyperglycemia is the chief hazard inducing microvascular endothelial dysfunction in the retina due to damage of pericytes. If injury due to high glucose is left untreated for longer period, other pathways in addition to glycolysis (namely hexosamine, polyol, and advanced glycation) get activated. These other pathways are recognized to induce apoptosis and degeneration of pericytes leading to the gradual damage to the retina.

The main contemporary interventions for Diabetic retinopathy (DR) or Diabetic macular edema (DME) are laser photocoagulation and vitrectomy (anti-vascular endothelial growth factor (anti-VEGF)), however, they are applied at advanced stages of disease only. Among those, vascular endothelial growth factor (VEGF) has been shown to be a critical stimulus in the pathogenesis of macular edema secondary to diabetes. Bevacizumab (Avastin) is a full-length antibody that inhibits all isoforms of the VEGF-A family. It is licensed by the US Food and drug administration FDA for the treatment of colorectal carcinoma. However, it is widely used in treatment of DME in form of intravitreal injections. However, despite good reduction in macular oedema after Bevacizumab injections, persistent macular edema or multiple remissions and exacerbations can result in foveolar photoreceptor damage with permanent impairment of vision. Intravitreal Avastin is not effective in reducing macular thickness after 4 weeks of injection. Injections like ranibizumab (Lucentis), aflibercept (Eylea) are also available, especially used in EU, but they also pose problems similar to Avastin. Implants such as Ozurdex (formerly Posurdex) which is FDA approved and is based on dexamethasone loaded polyglycolic acid (PLGA) matrix has been used clinically for the treatment of macular edema and non-infectious uveitis. However, complications have been reported in implantation and migration of the implant from the vitreous to the anterior chamber. However, all ophthalmic interventions are uncomfortable to the patients and result in side effects, when administered to a sensitive organ like that of eye. However, there are no oral compositions for intervention of Diabetic retinopathy.

In addition to Diabetes, the micro & macro vascular complications occur in other illnesses & its complications like Hypertension, Cardiac Disorder and more.

Gold, an inorganic material is used in herbal compositions owing to its biocompatibility, antioxidant and antiangiogenic properties and its ability to inhibit angiogenic molecules. Though gold has been used in several forms for various disorders, functionalization of gold nanoparticles by amorphous inorganic matrix and its activity in ophthalmic conditions while being administered orally is not studied at length.

Another inorganic compound that is widely used in herbal and vitamin compositions is Zinc. However, the activity of Zinc in conjunction with other components of herbal composition, other than vitamins is not studied extensively. Also, to the best of the knowledge of the present inventors, the surface functionalization of zinc is hitherto unknown.

Hence, there is a need in the art for an oral composition based out of natural products such herbs and minerals for the treatment of micro & macro vascular complications including Diabetic Retinopathy/Diabetic Macular edema, which can overcome the problems of the prior art.

### OBJECT OF THE INVENTION

A novel orally administered combination of bio-gold and bio-zinc, a process for its synthesis, a pharmaceutical composition comprising the combination and its applications, thereof in micro and macro vascular complications.

### SUMMARY OF THE INVENTION

The present invention is drawn to a combination (hereinafter also referred to “NTGX-10”) for oral administration for its utility in micro & macro vascular complications including Diabetic retinopathy or Diabetic macular edema. The present invention comprises a specifically surface functionalized nanoparticulate combination of bio-gold and bio-zinc. The bio-gold and bio-zinc of the present invention are structurally defined, safe, orally administered, bioprocessed. The present invention also discloses a process for obtaining the bio-gold and bio-zinc of the present invention, the combination of the present invention and a composition comprising the combination of the present invention along with pharmaceutically acceptable excipients. The present invention also discloses the utility of the combination and composition as disclosed herein for its effect in treatment of micro and macrovascular complications including diabetic macular edema (DME).

### BRIEF DESCRIPTION OF THE DRAWINGS

FIG. 1 depicts XRD pattern of Gold processed as per the present invention.

FIG. 2 depicts FESEM images of Gold based sample at magnifications of (a) 1000×, (b) 10000× and (c) 20000×.

FIG. 3 depicts FETEM images of Gold based sample at (a) low, (b) intermediate and (c) high magnification.

FIG. 4 depicts FETEM-STEM-HAADF-EDX elemental mapping images of Gold based formulation sample: (a) electron image, (b) AuL_{α1}, (c) C K_{α1-2}, (d) O K_{α1}, (e) Mg K_{α1-2}, (c) Ca K_{α1}, (g) layered image and (h) EDX spectrum.

FIG. 5 depicts XPS spectra of Gold based sample: (a) & (b) Survey scan and High Resolution scans corresponding to, (c) C 1s, (d) Au 4f, (e) O 1s, (f) Si 2p, (g) N 1s and (h) P 2p. XPS at (a) & (b) are obtained at SKKU and NCL, respectively.

FIG. 6 depicts XRD pattern of Zinc based formulation sample.

FIG. 7 depicts FESEM images of Zinc based sample at various magnifications (a) 1000×, (b) 10000× and (c) 20000×.

FIG. 8 depicts FETEM images of Zinc based sample at (a) low, (b) intermediate and (c) high magnification.

FIG. 9 depicts FETEM-STEM-HAADF-EDX elemental mapping images of Zinc based formulation sample: (a) electron image, (b) Zn K_{α1}, (c) O K_{α1}, (d) Si K_{α1}, (e) layered image and (f) EDX spectrum.

FIG. 10 depicts XPS spectra of Zinc based formulation sample: (a, b) Survey scans and High Resolution scans corresponding to (c) C 1s, (d) Zn 2p, (e) O 1s, and (f) Si 2p.

FIG. 11 depicts Effect on visual acuity after (a) oral NTGX-10 and (b) intravitreal bevacizumab administration.

FIG. 12 depicts Effect of oral NTGX-10 administration on contrast sensitivity.

FIG. 13 depicts OCT image at baseline showing presence of Maccular Edema (Case Study 1).

FIG. 14 depicts OCT image at Day 180 showing resolution of Maccular Edema (Case Study 1).

FIG. 15 depicts OCT image of Left Eye at baseline showing presence of Maccular Edema (Case Study 2).

FIG. 16 depicts OCT image of Left Eye at Day 60 showing reduction in Maccular Edema (Case Study 2).

FIG. 17 depicts OCT image of Left Eye at Day 120 showing resolution of Maccular Edema (Case Study 2).

FIG. 18 depicts OCT image of Left Eye at Day 180 showing resolution of Maccular Edema (Case Study 2).

FIG. 19 depicts OCT image of Left Eye at baseline showing presence of Maccular Edema (Case Study 3).

FIG. 20 depicts OCT image of Left Eye at Day 60 showing resolution of Maccular Edema (Case Study 3).

FIG. 21 depicts OCT image of Left Eye at Day 120 showing resolution of Maccular Edema (Case Study 3).

FIG. 22 depicts OCT image of Left Eye at Day 180 showing resolution of Maccular Edema (Case Study 3).

FIG. 23 depicts OCT image of Right Eye at baseline showing presence of Maccular Edema (Case Study 4).

FIG. 24 depicts OCT image of Right Eye at Day 120 showing reduction of Maccular Edema (Case Study 4).

FIG. 25 depicts images of the YSM model captured 24 h after the treatment with selected doses of NTGX10, Bioprocessed Gold and Bioprocessed Zinc.

FIG. 26 depicts counting method of blood vessels.

FIG. 27 depicts YSM 24h after the treatment with selected doses of NTGX-10, Bioprocessed Gold and Bioprocessed Zinc. Results of the counting of blood vessels in individual YSM in the form of an excel sheet are expressed.

FIG. 28 depicts graphical representation of the results carried out using sigma plot software.

FIG. 29 depicts comparative study of effect of bioprocessed gold, bioprocessed zinc and NTGX-10 (combination of Bioprocessed gold and zinc) on angiogenesis in experimental chick embryo YSM.

### DETAILED DESCRIPTION OF THE INVENTION

The present invention, is drawn to a novel orally administered combination of bio-gold and bio-zinc, a process for its synthesis, a pharmaceutical composition comprising the combination and its applications, thereof in micro and macro vascular complications.

The present invention is drawn to a combination of bio-gold and bio-zinc for oral administration for its utility in alleviating micro & macro vascular complications.

### Characteristics of the Combination of the Present Invention

The combination of the present invention has been extensively characterized and comprises the following parameters.

### Bio-Gold of the Present Invention

The XRD results reveal the presence of highly crystalline face-centered cubic (FCC) phase of gold. No other significant crystalline peaks attributable to Gold Oxide or other metallic impurities and/or their oxides are observed.
FESEM imaging shows formation of submicron to micron sized irregular shaped chunks with size varying from 500 nm to 10 microns which are further made up of agglomerated particles ranging from 100 nm to 500 nm. Also, gold nanoparticles are surrounded by the amorphous inorganic matrix.
FETEM images display the formation of polydispersed irregular shaped or regular shaped particles selected from group consisting of triangular, rectangular, rod-like, and spherical particles and their mixtures thereof composed of amorphous metal or amorphous metal oxides or mixtures.
FETEM-STEM-HAADF-Elemental Mapping images display the formation of a matrix of irregular shaped and regular shaped (triangular, rectangular, rod like, etc.) particles having faceted growth and spherical particles surrounding the predominantly spherical gold nanoparticles.
XPS study discloses peaks related to elemental gold (Au⁰) as well as peaks due to partial surface oxidation of gold having stable oxidation state of Au¹⁺ with a significant fraction (almost 60%) of oxidized Au¹⁺ species on the gold nanoparticles surface.

The combination of the present invention comprises the bio-gold in highly crystalline face-centered cubic (FCC) phase of gold.

The bio-gold of the present invention is present as a nano particulate system with bio-gold as spherical or distorted spherical particles in the core of the system. The nanoparticulate spherical or distorted spherical bio-gold is surrounded by amorphous inorganic matrix, of polydispersed irregular shaped or regular shaped particles selected from group consisting of triangular, rectangular, rod-like, and spherical particles and their mixtures thereof composed of amorphous metal or amorphous metal oxides or mixtures thereof in the particle-size range of 500 nm to 10 microns. The bio-gold comprises peaks related to elemental gold (Au⁰) and oxidated gold Au¹⁺ in the ratio of 2:3.

### Bio-Zinc of the Present Invention

XRD reveals formation of hexagonal phase of Zinc Oxide (ZnO). The characteristic peak of zinc metal is not noticed. FESEM images display formation agglomerated, heterogenous polydispersed irregular shaped or regular shaped particles selected from group consisting of triangular, rectangular, rod-like, and spherical particles and their mixtures thereof composed of amorphous metal or amorphous metal oxides or mixtures thereof in the particle-size range of 10 nM to 1000 nM.
FETEM images also disclose formation of submicron as well as nanoscale particles leading to significant polydispersity. Most of the particles exhibit faceted growth having, sheet-like, rod-like and irregular morphological features, with few spherical particles.
XPS scans reveal presence of peaks due to elements such as Zn, O, Mg, Si, C, K, Ca, etc., implying that MgO, SiO₂, CaO, etc. are forming the amorphous matrix around submicron scale particles and nanoparticles of ZnO.

The bio-zinc is present as hexagonal phase of Zinc Oxide (ZnO) and without any free Zinc. The bio-zinc is present as agglomerated, heterogenous polydispersed irregular shaped or regular shaped particles selected from group consisting of triangular, rectangular, rod-like, and spherical particles and their mixtures thereof composed of amorphous metal or amorphous metal oxides or mixtures thereof in the particle-size range of 10n M to 1000 nM. The bio-zinc comprises additional elements selected from the group consisting of Zn, O, Mg, Si, C, K, Ca, or combinations thereof. The bio-zinc comprises a coating of MgO, SiO₂, CaO, are forming the amorphous matrix around submicron scale particles and nanoparticles of ZnO.

The combination of the present invention comprises bio-gold in the range 2 to 4% (w/w); bio-zinc in the range of 3 to 5% (w/w) and in the ratio of 1:1 to 1:4.

The process of the present invention comprises the step of obtaining the bio-gold comprising the steps of:
i. Obtaining metallic Gold of purity 99.99% in the form of foils/powder/flakes,
ii. heating gold of step (i) until the gold is red hot,
iii. quenching the red-hot gold of step (ii) in the aqueous extract of rhizome of Curcuma longa,
iv. carrying out steps (ii) and (iii) in cycles for 10 to 100 times, preferably 15 to 50 times, more preferably for 20 to 40 times to obtain a brittle gold,
v. triturating the brittle gold of step (iv) with the aqueous extract of rhizome of Curcuma longa in the ratio of 1:1 to 1:2 for a period of 6-8 hrs to obtain a powder,
vi. placing the powder in a covered earthen vessel and heating the mixture for 100° C. to 1000° C., preferably 350° C. to 850° C., more preferably to 450° C.-750° C. for a period of 20-45 min. and then allowed to cool gradually to room temperature,
vii. carrying out steps (v) and (vi) in cycles for 10 to 500 times, preferably 50 to 250 times, more preferably for 80 to 120 times to obtain a finely divided pinkish colored final bio-gold in the particle size ranging from submicron to nano scale.

The process of the present invention comprises the step of obtaining the bio-zinc comprising the steps of:
i. obtaining metallic Zinc of purity 99.99% in the form of foils/powder/flakes,
ii. heating of zinc of step (i) till it completely liquifies,
iii. Quenching the liquefied zinc of step (ii) in the aqueous extract of the pulp of the fruits Phyllathus embellica after removing the seeds,
iv. carrying out steps (ii) and (iii) in cycles for 10 to 100 times, preferably 15 to 75 times, more preferably for 20 to 40 times to obtain a brittle zinc,
v. triturating the brittle zinc of step (iv) with the aqueous extract of the pulp of the fruits Phyllathus embellica in the ratio of 1:1 to 1:2 for a period of 6-8 hrs to obtain a powder,
vi. Placing the powder in a covered earthen vessel and heating the mixture for 100° C. to 1000° C., preferably 250° C. to 750° C., more preferably to 350° C.-600° C. for a period of 20-45 min. and then allowed to cool gradually to room temperature,
vii. Carrying out steps (v) and (vi) in cycles for 10 to 100 times, preferably 20 to 80 times, more preferably for 30 to 60 times to obtain a finely divided powder of bio zinc of the present invention.

The process of obtaining the combination of the present invention by combining bio-nano-gold and the bio-nano-zinc in the ratio of 1:1 to 1:4.

In an aspect the present invention discloses a composition comprising the combination of the present invention as disclosed herein along with pharmaceutically acceptable excipients.
i. A composition comprising bio-gold in the range 2 to 4% (w/w); bio-zinc as in the range of 3 to 5% (w/w) and pharmaceutically acceptable excipients in the range of 91 to 95% (w/w).

The combination or composition may be administered as an oral dosage form in a dose of 0.02-2.00 mg/Kg.

The administration of the composition of the present invention can be by an oral dosage form, preferably in unit dosage forms suitable for simple administration of precise dosages. Solid dosage forms for oral administration include capsules, tablets, pills, powders, and granules. Solid dosage forms, as described above, can be prepared with coatings and shells, such as enteric coatings. Liquid dosage forms for oral administration include pharmaceutically acceptable emulsions, solutions, suspensions, syrups, and elixirs.

In an embodiment, the combination of the present invention may be administered orally for its use in micro and macro vascular complications including diabetes, diabetes induced ophthalmic disorders such as Diabetic retinopathy and Diabetic macular edema.

The combination and the composition comprising the combination of the present invention may be utilised in the following complications that may stem out of macro or micro vascular complications.
1. Coronary Heart Disease
2. Myocardial ischemia
3. Myocardial Infraction
4. Neuropathy
5. Nephropathy
6. Retinopathy
7. Dementia
8. Stroke
9. Chronic Kidney Failure
10. Erectile Dysfunction
11. Peripheral Vascular Disease
12. Vasculitis as follows:
   Large vessel: Takayasu's arteritis, Temporal arteritis
   Medium vessel: Buerger's disease, Kawasaki disease, Polyarteritis nodosa
   Small vessel: Behcet's syndrome, Eosinophilic granulomatosis with polyangiitis, Cutaneous vasculitis, granulomatosis with polyangiitis, Henoch-Schönlein purpura, and microscopic polyangiitis
13. Venous diseases as follows:
   Deep vein thrombosis
   Superficial venous thrombosis or phlebitis
   Chronic venous insufficiency
   Varicose and spider veins

In other words NTGX-10 is meant to attenuate the adverse effects of systemic illness on blood vessels.

The combination or composition of the present invention may be administered as an oral dosage form in a dose of 0.02-2.00 mg/Kg for its utility in alleviating micro and macrovascular complications, in diabetic macular edema (DME), in Diabetic retinopathy.

The combination or composition of the present invention is capable of acting in mammals independent of pathology.

### Advantages of the Combination and the Composition of the Present Invention

1. The combination of the present invention (NTGX-10) has neuroprotective effect against toxicity induced by anticancer drugs in vivo, has several advantageous effects as below in addition to its ophthalmological action.
2. NTGX-10 is orally administrable and has a ease of administration unlike other drugs used in treatment of diabetic retinopathy which needs intravitreal administration.
3. NTGX-10 is neuroprotective and helps protect the retina against the ill effects of systemic illness like hypertensin, diabetes and metabolic syndrome.
4. NTGX-10 is well tolerated and safe even in long term use.

All biological resources of the present invention are readily commercially available and were procured from the local market.

The present invention is illustrated by means of the examples. The Examples are meant only for illustration and cannot be construed as limiting.

### Example 1: Process for Preparation of the Composition of the Present Invention

### Bioprocessing of Gold

### Step 1:

Metallic Gold of purity 99.99% is taken in the form of foils/powder/flakes. This pure metallic gold has been exposed to heat till it becomes red hot and dipped in the aqueous extract of Curcuma longa. This metal is allowed to cool down within the aqueous extract and then removed from the extract. This process is repeated in the same manner for minimum 21 times. This process results in making the metal softer and brittle than its original nature. In the end, the metallic gold is removed from aqueous extract of Curcuma longa.

### Step 2:

Material received from step 1 is again mixed with the aqueous extract of Curcuma longa. This mixing can be done by trichuration for 6-8 hrs or any other suitable method. After this the mixture is then kept in an earthen vessel sealed muslin cloth. This earthen vessel is then exposed to heat to about 350° C.-600° C. but not limited to this range only. This exposure of heat is given in a gradually increasing manner and kept at about 600° C. for 20-45 min. and then allowed to cool gradually to room temperature.

The whole process of trichuration and then exposure to heat is repeated for 80-120 times till a finely divided pinkish colored material is formed. This final material should not contain any traces of metallic gold and should have the particle size ranging from submicron to nano scale.

### Bioprocessing of Zinc

### Step 1:

Metallic Zinc of purity 99.99% is taken in the form of foils/powder/flakes. This pure metallic zinc has been exposed to heat till it liquifies and is dipped in the aqueous extract of Phyllathus embellica. This metal is allowed to cool down within the aqueous extract and then removed from this extract.

This process is repeated in the same manner for minimum 21 times. This process results in making the metal softer and brittle than its original nature. In the end, the metallic zinc is removed from the aqueous extract of Phyllathus embellica.

### Step 2:

Material received from step 1 is again mixed with the aqueous extract of Phyllathus embellica. This mixing can be done by trichuration for 6-8 hrs or any other suitable method. After this, the mixture is kept in an earthen vessel sealed muslin cloth. This earthen vessel is then exposed to heat to about 400° C.-800° C. but not limited to this range only. This exposure of heat is given in a gradually increasing manner and kept at about 600 0 C for 20-45 min. and then allowed to cool gradually to room temperature.

The whole process of trichuration and then exposure to heat is repeated for 80-120 times till a finely divided material is formed. This final material should not contain any traces of metallic zinc and should have the particle size ranging from submicron to nano scale.

### Example 2: Characterisation of the Product of the Present Invention

Since NTGX-10 based formulations are biologically processed metallic (Au, Zn) materials, the present invention utilises relevant characterization techniques which are non-destructive in nature. It was necessary to determine the characteristics of the material in its pristine state.

### Example 2.1; Characterization of the Bio-Gold of the Present Invention

### 2.1.1. X-Ray Diffraction (XRD)

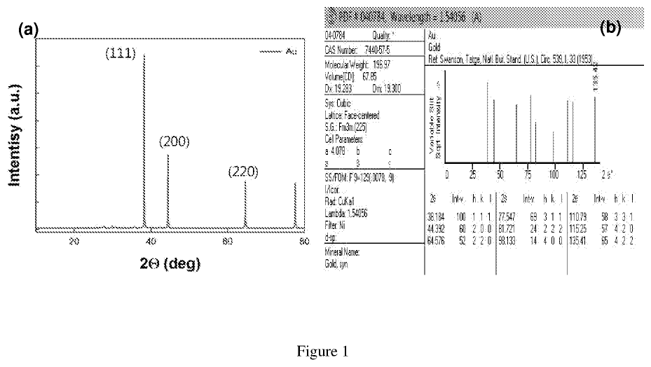

XRD pattern of Bio-gold based formulation recorded by using X-ray diffractometer (Bruker, D8, ADVANCE, Germany) with Ni-filtered CuKα radiation (λ=1.54 Å) is shown in FIG. 1. The XRD pattern matches well with the JCPDS file no. 04-0784 corresponding to the face centered cubic (FCC) phase of gold. The peaks corresponding to (h k 1) planes of (1 1 1), (2 0 0), (2 2 0) and (3 1 1) can be clearly seen. The peaks are very sharp implying highly crystalline nature of the sample. Such nature can be the obvious consequence of carrying out the process at high temperature around 700-800° C. repeatedly. No other significant crystalline peaks attributable to Gold Oxide or other metallic impurities and/or their oxides (arising from matrix composition and other added ingredients involved during preparation) are observed in the sample.

### 2.1.2 Field Emission Scanning Electron Microscopy (FESEM)

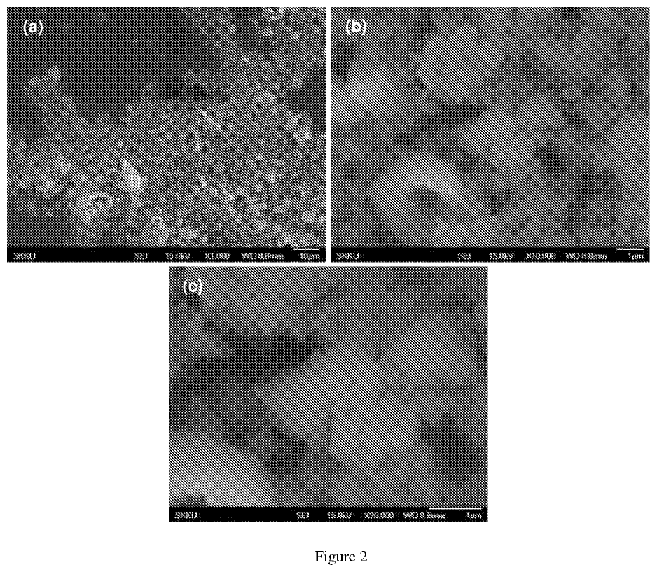

FESEM images of the Bio-gold based formulation sample were obtained with the instrument JSM6700F (JEOL, Japan). For this purpose, the powdered sample was mounted on aluminum stub. FESEM images of the Bio-gold based formulation sample at different magnifications (1000×, 10000× and 20000×) are shown in FIG. 2. Low magnification image shows formation of submicron to micron sized irregular shaped chunks (FIG. 2a). The size of such chunks varies from 500 nm to 10 microns. The intermediate magnification image (FIG. 2b) reveals that such chunks are made up of agglomerated particles. The high magnification image discloses (FIG. 2c) that size of the agglomerated particles ranges from 100 nm to 500 nm. Owing to secondary electron (SE) mode morphology imaging and also on account of charging effect occurred in case of this sample, not much significant information can be obtained. However, it can be inferred from this charging phenomenon that metallic nanoparticles are surrounded by the layer/coating/matrix of inorganic ingredient, probably metal oxide particles.

### 2.1.3 Field Emission Transmission Electron Microscopy (FETEM)

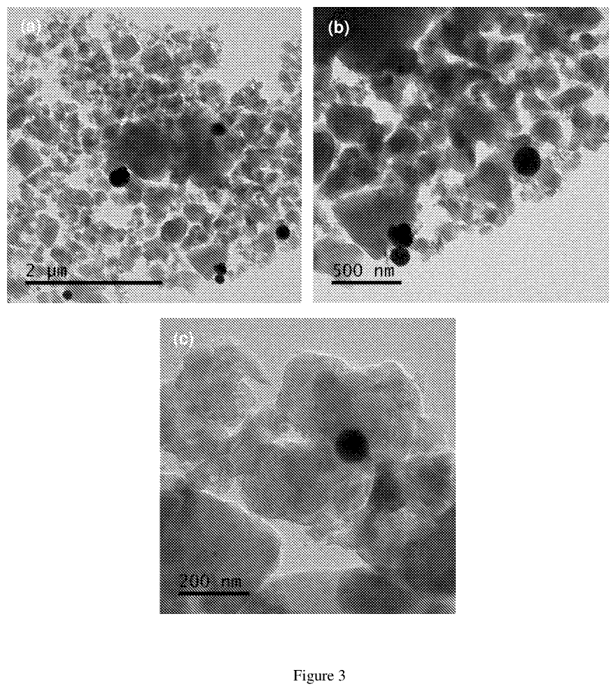

Fine-scale microstructural evaluation of the Bio-gold based formulation sample was accomplished by using FETEM (JEOL, USA, JEM ARM 200F). For FETEM analysis, the test sample was carefully prepared by dispersing the sample powder in de-ionized water and drop of the dispersion was then transferred to carbon coated grid. FETEM images of Bio-gold based formulation sample at low, intermediate and high magnification are shown in FIGS. 3a, b and c, respectively. Quite interestingly, the low magnification FETEM image (FIG. 3a) reveals the formation of micron (occasional), submicron, as well as nanoscale particles. Some of the particles show faceted growth, while others show irregular shapes and few spherical particles are also seen. Some of the particles exhibit strong atomic number contrast which may be ascribed to the presence of gold as gold is heavier than other constituents of the Bio-gold based formulation (please see subsequent sections). Intermediate magnification FETEM image (FIG. 3b) also reveals the formation of a matrix of irregular shaped and regular shaped (triangular, rectangular, rod like, etc.) particles having faceted growth and spherical particles surrounding the gold nanoparticles. Though most of the gold nanoparticles have spherical shape, intermittently irregular shaped particles are also noticed. The high magnification FETEM image (FIG. 3c) displays a representative gold nanoparticle surrounded by a matrix consisting probably of amorphous constituents of the formulation.

### 2.1.4 STEM-HAADF Tomography with Elemental Mapping and EDX

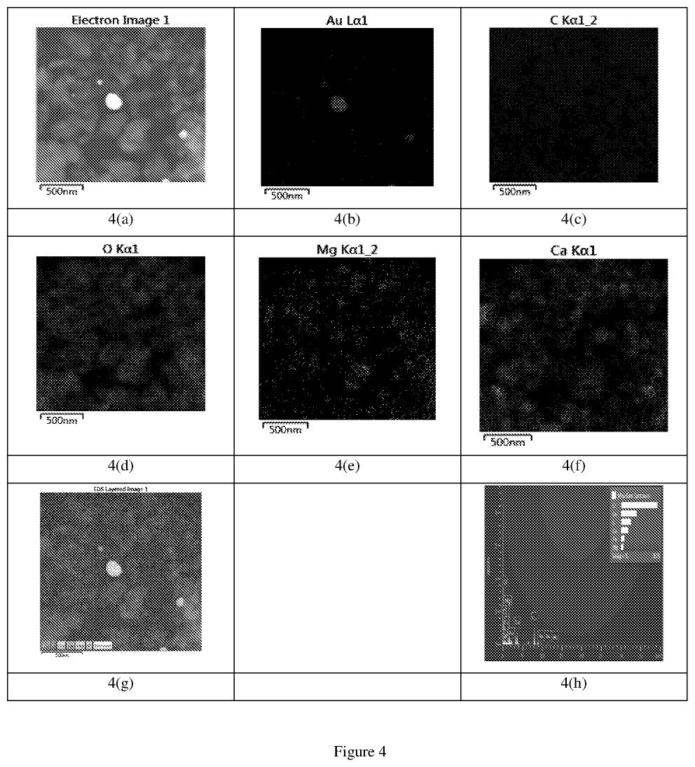

In order to acquire high imaging resolution and spatial resolution for atom to atom chemical mapping of the material, Scanning Transmission Electron Microscopy (STEM) imaging equipped with High Angle Annular Dark-Field Detector (HAADF) and Energy Dispersive X-Ray Spectroscopy (EDS) Elemental Mapping by using JEOL, JEM ARM200F equipment operated at 200 kV with spherical aberration corrector was carried out. The resultant STEM-HAADF tomography image with elemental mapping images and EDX data for Bio-gold based formulation sample are furnished in FIG. 4.

The STEM-HAADF image reveals nanoscale particles with dark atomic contrast embedded in the matrix of submicron and nanoscale particles with light atomic contrast (FIG. 4a). The darker particles can be assigned to gold (FIG. 4b) while lighter ones can be assigned to the matrix consisting of other major constituents identified as carbon, oxygen, magnesium, calcium, etc. (FIG. 4c-e). The elemental mapping (FIG. 4b-e) further substantiates this observation. Overlaying of mapping images of calcium and magnesium with oxygen hints the formation of MgO and CaO as the major constituents of the matrix. However, their presence is not revealed in the XRD which may be due to their amorphous nature or low crystallinity on the global scale. Surprisingly, oxygen is also seen to be present at the dark particles assigned to gold albeit with low intensity. This may be attributable to the partial oxidation of the gold surface which might be the consequence of the critical conditions of Bio-gold based formulation preparation sequence.

Table 1 illustrates the elemental distribution of the constituents of the Bio-gold based formulation sample as obtained from the EDS analysis.

TABLE 1
Weight to atomic percentage in EDS of
Bio-gold based formulation sample.

| | | Approximate Weight | Approximate Atomic |
|---|---|---|---|
| Sr No. | Element | % | % |
| 1. | C | 24 | 35.66 |
| 2. | O | 48 | 53.53 |
| 3. | Au | 10 | 0.91 |
| 4. | Ca | 14 | 6.23 |
| 5. | Mg | 5 | 3.67 |

### 2.1.5 X-Ray Photoelectron Spectroscopy (XPS)

It may be noted that while EDS offers only the elemental composition of the chemical compound, XPS specifies the exact chemical composition of the compound although confined to surface/sub-surface area. To investigate the surface chemical composition of the Bio-gold based formulation sample, X-ray photoelectron (XP) spectra were recorded on VG MicroTech ESCA 3000 instrument at a pressure better than 1×10⁻⁹ Torr. The survey scan and C 1s, Au 4f and O 1s core level spectra were recorded with monochromatized Mg K_{α} radiation (photon energy=1253.6 eV) at the pass energy of 50 eV and electron take-off angle (angle between electron emission direction and surface plane) of 60 degrees. The overall resolution of measurement is thus ˜1 eV for the XPS analysis. The core level spectra were subjected to background-correction using the Shirley algorithm and were aligned with respect to the adventitious C 1s BE of 285 eV (Table 2).

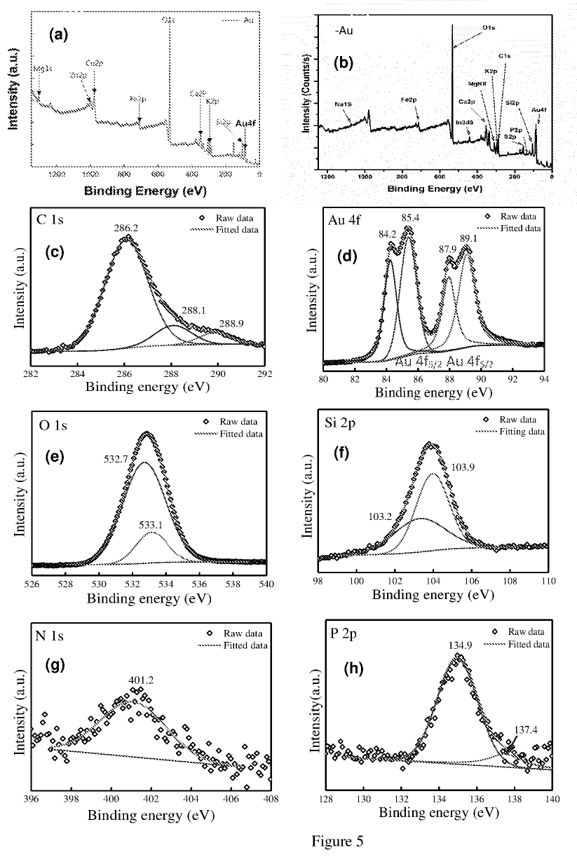

The surface composition of prepared Bio-gold based formulation sample was examined by XPS. FIG. 5 comprises the XPS survey scans as well as high resolution spectra for carbon, gold, oxygen, silicon, nitrogen and phosphorous. The XPS survey scans revealed presence of carbon, gold, oxygen, silicon, magnesium, calcium, nitrogen and phosphorous etc. FIG. 5d shows a high resolution spectrum of the Au 4f core level. This spectrum displays two major pairs of duplet peaks. Each duplet pair might be due to spin-orbit coupling (Au4f_{7/2} and Au4f_{5/2}). From first and second peaks, the binding energy (BE) values of 84.2 and 87.9 eV respectively are related to elemental gold (Au⁰). Quite interestingly, the peaks corresponding to binding energy of 85.4 eV and 89.1 eV are slightly more intense than the peaks due to elemental gold. These peaks are attributable to partial surface oxidation of gold having stable oxidation state of Au¹⁺. Their relative distribution (area under the curve) discloses a significant fraction (almost 60%) of oxidized Au¹⁺ species on the gold nanoparticles surface.

It is important to note that XPS is known to be a technique applied to probe relatively thin near-surface layers (emerging electrons come from the first few nanometers under the surface), while inner metallic layers may not contribute to the integral XPS signal. Therefore, Au¹⁺ can mainly be assigned to the partial surface oxidation (at this juncture) hinting the formation of Au₂O. There is a probability of bulk oxidation of nanoscale gold. However, it can be ruled out in view of the following facts:
1. Gold is reported to be an inert noble metal. Therefore, it is not chemically active, except at the surface, especially at the nanoscale. Surface chemical reactivity of nanoscale gold is well documented. Even partial oxidation of gold at surface is reported. However, it can only be determined by XPS and not by XRD technique.
2. A bulk characterization technique, XRD, revealed presence of peaks in the sample due to metallic gold only (Phase Purity). Diffraction Peaks due to other impurities (especially arising from matrix compounds) are not seen in the XRD pattern corresponding to bio-gold.

TABLE 2
XPS BE, atomic and Wt % for bio-Gold based formulation sample.

| Name | Start BE | Peak BE | End BE | Atomic % | Wt % |
|---|---|---|---|---|---|
| MgKLL | 314.08 | 307.76 | 301.08 | 12.12 | 11.69 |
| O1s | 538.58 | 533.09 | 523.08 | 43.95 | 27.89 |
| Au4f | 98.08 | 85.72 | 80.58 | 2.27 | 17.73 |
| Si2p | 110.08 | 104.04 | 94.58 | 11.47 | 12.74 |
| C1s | 292.08 | 286.77 | 281.08 | 14.88 | 7.07 |
| K2p | 301.58 | 295.52 | 292.08 | 2.3 | 3.53 |
| Ca2p | 359.08 | 353.08 | 344.08 | 5.35 | 8.48 |
| In3d5 | 446.58 | 441.39 | 439.58 | 0.15 | 0.66 |
| Fe2p | 738.58 | 713.09 | 702.58 | 2.26 | 5.02 |
| Na1s | 1077.58 | 1073.37 | 1061.58 | 1.25 | 1.12 |
| P2p | 139 | 135.38 | 125 | 1.05 | 1.29 |
| S2p1 | 170 | 165.88 | 159 | 1.05 | 1.31 |
| S2p3 | 170 | 165.88 | 159 | 0.54 | 0.669 |
| N1s | 408 | 396.37 | 394 | 1.35 | 0.74 |

O1s is the most intense peak in both the Survey Scans (FIG. 5 a & b). XPS % analysis reveals atomic % value of 43.95 for chemically active oxygen species which is reasonably higher than atomic percentage value for oxygen species obtained from EDS analysis in FETEM (31.8%). However, reverse trend is observed in weight % values obtained by XPS (27.89%) and by EDS (48%), (Refer to Tables 1&2). Atomic % of Au is 2.27% by XPS while it is around 0.27% by EDS. Weight % values for Au obtained by XPS (17.73%) and EDS (5%) also indicate similar trend.

TABLE 3
XPS BE values and corresponding possible assignment
for Bio-gold based formulation sample.

| Name | Peak BE | Possible Assignment |
|---|---|---|
| O1s | 532.7 | Surface Oxide |
| | 533.1 | Sub-surface Oxide |
| Au4f | 82.4 | Metallic Au |
| Au4f | 85.4 | Au in the composition of oxide/Au2O3 |
| Au4f | 87.9 | Metallic Au |
| Au4f | 89.1 | Au in the composition of oxide/Au2O3 |

XPS findings summarized in Table-3 are perplexing and can be associated with presence of chemisorbed and/or sub-surface adsorbed oxygen species on nano-Au along with strong possibility of partial oxidation of Au at surface/sub-surface level mediated through chemisorbed/adsorbed oxygen species. It should be noted that XPS is surface sensitive technique with limitation to detect bulk composition. It is inferred that chemically active oxygen can mask bulk detection of Au in the Au based formulation samples. It might be applicable to EDAX technique to some extent. Probing the bulk-level partial oxidation of gold can be achieved with He-ion microscopy equipped with electron energy loss spectroscopy (EELS).

### Example 2.2; Characterization of the Bio-Zinc of the Present Invention

### Example 2.2.1; X-Ray Diffractometry (XRD)

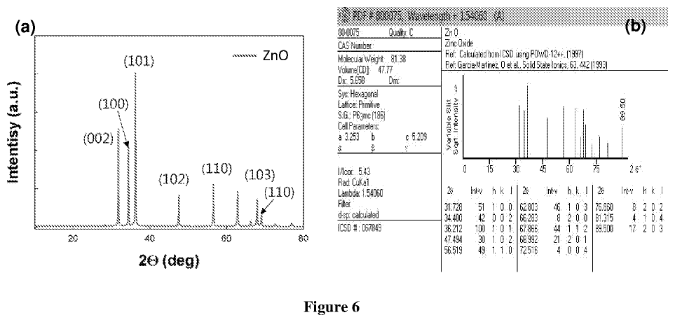

XRD technique is an useful tool for identifying the presence of the crystalline phases in the sample. XRD pattern of Bio-zinc based formulation recorded by using X-ray diffractometer (Bruker, D8, ADVANCE, Germany) with Ni-filtered CuK_{α} radiation (λ, =1.54 A) is shown in FIG. 6. It reveals the formation of hexagonal phase of Zinc Oxide (ZnO) when identification was done by matching d spacing with the standard JCPDS database (CARD NO 80-0075). Peaks at d=2.47 Å (2Θ=) 36.25°, d=2.81 Å (2Θ=) 31.75°, and d=1.62 Å (2Θ=) 56.58° confirm the presence of ZnO (hexagonal) as the major crystalline phase in the sample. Seven most intense reflection peaks of the sample correspond to (101), (100), (002), (110), (102), (103), (110) planes of hexagonal ZnO (lines in standard data). The presence of starting material in the preparation of Bio-zinc based formulation (crystalline metallic zinc sheet) with the characteristic peak of zinc metal appearing at d=2.09 Å (2Θ=) 43.23° is not noticed. This result confirms the absence of any crystalline zinc metal in the final product of the Bio-zinc based formulation.

### Example 2.2.2 Field Emission Scanning Electron Microscopy (FESEM)

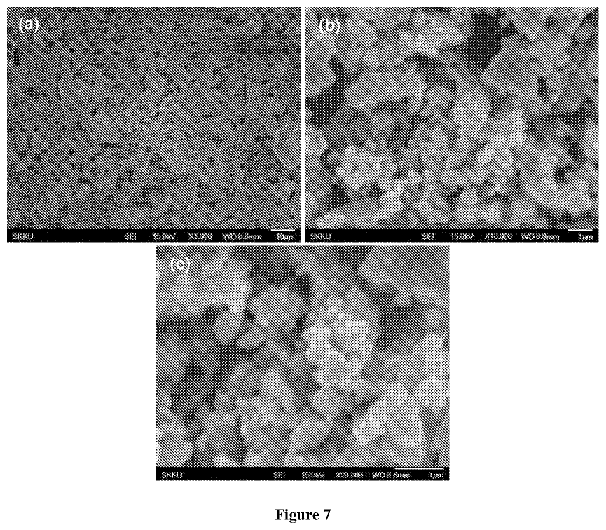

FESEM examination is an important step in determining the size and morphological features of the solid material. FESEM images of the Bio-zinc based formulation sample were obtained with the instrument JSM6700F (JEOL, Japan). For this purpose, the powdered sample was mounted on aluminium stub. Typical FESEM images of the Bio-zinc based formulation sample are shown in FIG. 7. These images display formation of agglomerated particles (FIG. 7a). The particles have various shapes such as sheets, rods, spherical as well as irregular particle shapes, etc. (FIG. 7b). Faceted growth is observed in most of the particles which is a common trend in case of ZnO nanoparticles due to their peculiar hexagonal crystal structure. Even though, maximum number of particles exhibit submicron particle size, large number of nanoparticles having size below 100 nm are also observed.

### Example 2.2.3 Field Emission Transmission Electron Microscopy (FETEM) and Scanning Transmission Electron Microscopy (STEM) with Elemental Mapping

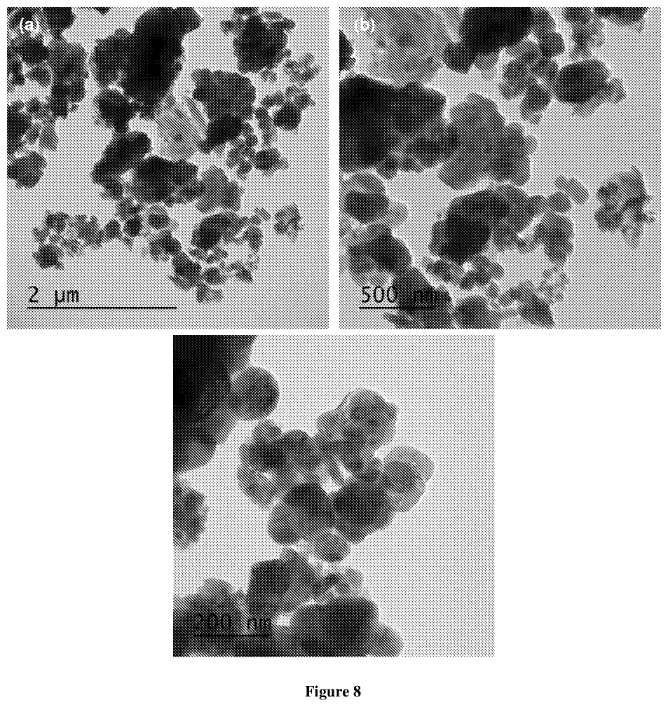

Fine-scale microstructural evaluation of the Bio-zinc based formulation sample was accomplished by using FETEM (JEOL, USA, JEM ARM 200F). For FETEM analysis, the test sample was carefully prepared by dispersing the sample powder in de-ionized water and drop of the dispersion was then transferred to carbon coated Cu grid. Typical FETEM images obtained at low, intermediate and high magnification are presented in FIGS. 8a, b and c, respectively. Like FESEM images, FETEM images also disclose formation of submicron as well as nanoscale particles leading to significant polydispersity. Most of the particles exhibit faceted growth having, sheet-like, rod-like and irregular morphological features.

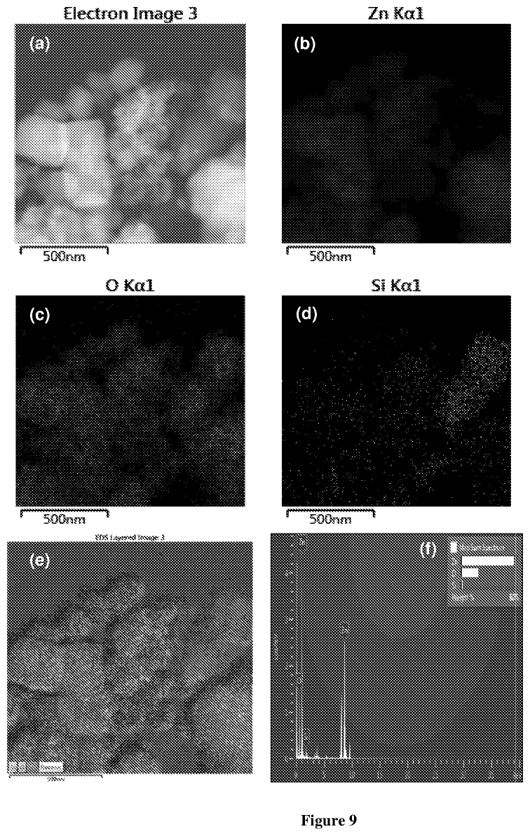

Intermittently, spherical particles are also seen. In order to acquire high imaging resolution and spatial resolution for atom to atom chemical mapping of the material, Scanning Transmission Electron Microscopy (STEM) equipped with High Angle Annular Dark-Field Detector (HAADF) and Energy Dispersive X-Ray Spectroscopy (EDS) Elemental Mapping by using JEOL, JEM ARM200F equipment operated at 200 k V with spherical aberration corrector was carried out. The resultant STEM-HAADF tomography image with elemental mapping images and EDX data for Bio-zinc based formulation sample are furnished in FIG. 9.

### Example 2.2.4 STEM-HAADF Tomography with Elemental Mapping and EDX

The STEM-HAADF image (FIG. 9a) shows atomic number contrast for zinc atom. However, this contrast is not very strong (as observed in case of Au based formulation samples). It is expected owing to the fact that the difference in atomic numbers of zinc, oxygen and silicon is not very high. In elemental mapping images (FIGS. 9b and c), the exact overlap of Zn K_{α1} element map with O K_{α1} implies formation of ZnO in close agreement with XRD data. Occasional presence of SiO₂ is also detected in the same manner (FIGS. 9c and d). Also, unlike Au based formulation sample, the weight percentage of zinc in the Bio-zinc based formulation sample is maximum (FIG. 9f). Besides, EDX mapping summary data indicates weight % values of 75% and 25% for Zn and O, respectively, (which correspond to atomic % values of 16.5% and 22.3% for Zn and O, respectively)

### Example 2.2.5 X-Ray Photoelectron Spectroscopy (XPS)

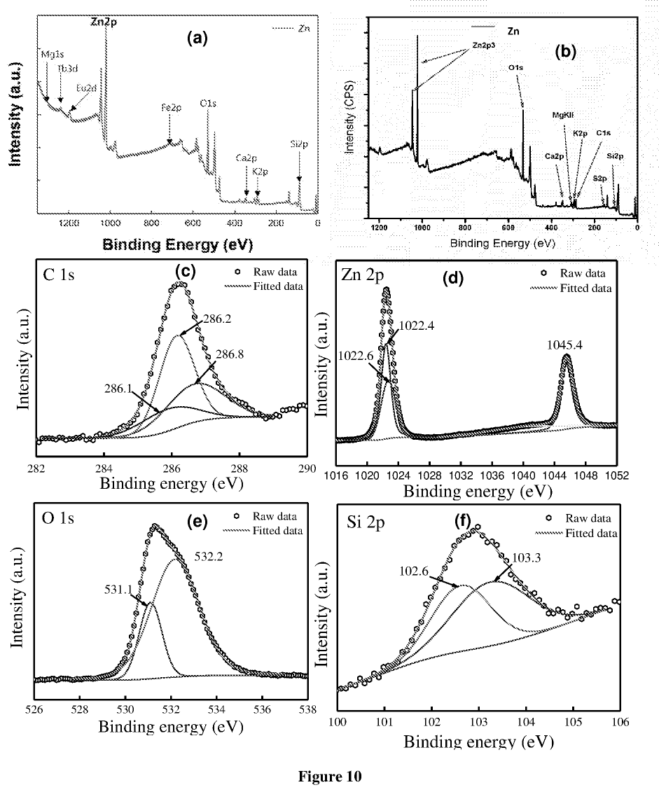

It may be noted that while EDS offers only the elemental composition of the chemical compound, XPS specifies the exact chemical composition of the compound although confined to surface/sub-surface area. To investigate the surface chemical composition of the Bio-zinc based formulation sample, X-ray photoelectron (XP) spectra were recorded at on VG MicroTech ESCA 3000 instrument at a pressure better than 1×10⁻⁹ Torr. The survey scan and C 1s, Zn 2p and O 1s core level spectra were recorded with monochromatized Mg K_{α} radiation (photon energy=1253.6 eV) at the pass energy of 50 e V and electron take-off angle (angle between electron emission direction and surface plane) of 60 degrees. The overall resolution of measurement is thus ˜1 eV for the XPS analysis. The core level spectra were subjected to background-correction using the Shirley algorithm and were aligned with respect to the adventitious C 1s BE of 285 ev. The chemically distinct species were resolved using a non-linear least squares procedure. XP spectra of Bio-zinc based formulation sample are shown in FIG. 10. XPS survey scan (FIG. 10a) reveals presence of peaks due to elements such as Zn, O, Mg, Si, C, K, Ca, etc. XPS survey scans correlate/superimpose well with each other. Peak BE positions of Zn2_{p3/2} and Zn2_{p1/2} in the XP spectra of Bio-zinc based formulation (displayed in FIG. 10a, d) are 1022.93 and 1045.4 eV respectively. Table 4 summarizes the details about the XPS BE, atomic and weight % values for the different elements present in the Bio-zinc based formulation sample. Table 5 provides probable BE assignments for the prominent XPS peaks which primarily reveals formation of ZnO with the indication of Zn (OH)₂. Atomic percentage values corresponding to Zn obtained from EDS analysis in FETEM (16.5%) and from XPS (15.7%) are in close agreement. However, there is mismatch of atomic percentage values corresponding to O obtained from EDS analysis in FETEM (22.3%) and from XPS (46.52%). It might be attributed to chemisorption of O species at the ZnO surface which can be detected by surface sensitive XPS tool.

TABLE 4
XPS BE, Atomic (At) and Weight (wt) % values
for Bio-zinc based formulation sample.

| Name | Start BE eV | Peak BE eV | End BE eV | Atomic % | Wt % |
|---|---|---|---|---|---|
| Mg_{KLL} | 310.08 | 306.72 | 303.08 | 8.7 | 8.14 |
| Si₂ₚ | 106.08 | 102.95 | 99.08 | 2.28 | 2.45 |
| Zn₂ₚ | 1029.58 | 1022.93 | 1015.08 | 15.75 | 39.65 |
| O₁ₛ | 538.58 | 532.21 | 527.58 | 46.52 | 28.66 |
| K₂ₚ | 300.08 | 295.1 | 292.08 | 3.66 | 5.49 |
| C₁ₛ | 292.08 | 286.5 | 279.58 | 17.78 | 8.2 |
| Ca₂ₚ | 358.58 | 348.27 | 342.58 | 2.78 | 4.26 |
| S₂ₚ₁ | 170 | 164.91 | 159 | 1.68 | 2.06 |
| S₂ₚ₃ | 170 | 164.91 | 159 | 0.86 | 1.05 |

TABLE 5
XPS BE values and corresponding possible assignment
for Bio-zinc based formulation sample.

| Name | Peak BE (eV) | Possible assignment |
|---|---|---|
| Zn2p_{3/2} | 1022.6 | Zn—O |
| Zn2p_{3/2} | 1022.4 | Zn—O |
| Zn2p_{1/2} | 1045.4 | Zn—O |
| O1s | 531.1 | Zn—O |
| O1s | 532.2 | Zn(OH)₂ |
| C₁ₛ | 286.1, 286.2 286.8 | Residual organic carbon |

### Example 3: Composition of the Present Invention

NTGX-10 is formulated with bioprocessed gold and bioprocessed zinc. Aqueous extracts of Phyllanthus emblica and Curcuma longa are used in the synthesis.

The combination of the present invention basic pathophysiological process involved in micro and macrovascular complications in DM, HT, etc. including Diabetic retinopathy by reducing the progression and complications involved in the disease. Majority of conventional treatment options for Diabetic retinopathy are meant to be used intravitreal i.e. injection in eye, however this particular product is meant to be used orally.

### Example 4: Biological Testing of the Composition of the Present Invention

### 4.1. Clinical Testing of the Combination of the Present Invention

### 4.1.1 Protocol for Clinical Testings

In a six month clinical study conducted on patients with diabetic retinopathy, the study participants orally consumed one capsule of NTGX-10 twice daily. Patients underwent baseline evaluation which included ophthalmic evaluations and safety evaluation. Haematological, Biochemical and ophthalmic investigations were performed as per the schedule of assessment to assess the efficacy and safety of the study drug.

Visual acuity, contrast sensitivity and slit lamp evaluation test were evaluated on monthly intervals. Fundus fluorescent angiography and OCT were performed every 2 months to access macular edema, hemorrhages and other ophthalmic complications. Hematological and biochemical evaluations along with fundoscopy, ophthalmoscopy and intra ocular pressure evaluation were performed after 90 and 180 days of treatment initiation. Patient reported outcomes were captured in NEI Visual Function questionnaire every month. Patients were provided with patient diary to monitor the medication compliance. Adverse events were monitored throughout the study.

In the analysis, the data pertaining to Best Corrected Visual Acuity (BCVA), Contrast sensitivity, macular oedema and need for intravitreal injections (as an indicator of efficacy) and all safety related variables like CBC, LFT, RFT were assessed. Besides this, secondary efficacy variables like HbA1C and Lipid profile are also analyzed to evaluate if the study formulation has any effect on these confounding variables.

### 4.1.2 Best-Corrected Visual Acuity (BCVA) Measurements

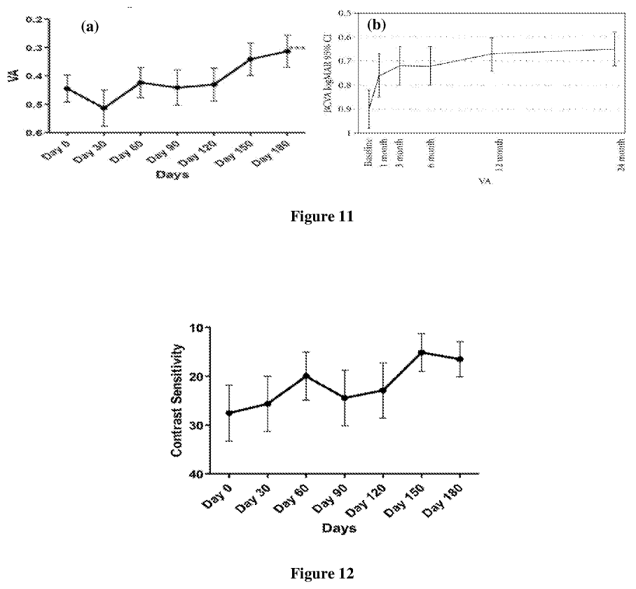

BCVA was checked in patients using Snellen's chart and converted to log MAR for statistical analysis. Improvement in visual acuity is indicative of improved visual functioning. Post 6 months NTGX therapy, a statistically significant improvement in BCVA was observed (0.42 to 0.31 a difference of 0.11 log MAR) (p<0.001) (FIG. 11b). A subset analysis of population with reduced BCVA (>0.7 Log MAR) (n=26 eyes) suggests statistically significant improvement in BCVA (1.01 to 0.77 a difference of 0.24 log MAR) (p<0.001). (FIG. 11b) In another study the changes in best-corrected visual acuity (BCVA) after intravitreal bevacizumab at 1 month (FIG. 11c) was from 0.90 to 0.76, a difference that was statistically significant (P<0.001)⁴.

### 4.1.3: Contrast Sensitivity in Patients

Contrast sensitivity (CS) is an important outcome measure and could be considered as an adjunct to standard VA testing for a more complete assessment of visual function in DME patients. In the present study, Contrast sensitivity was evaluated at monthly intervals. Statistically significant (p<0.01) improvement in CS was observed after 180 days of NTGX-10 treatment (FIG. 12).

### 4.1.4: Efficacy Evaluation in Patients

In a reported clinical study conducted in 54 Irish patients of diabetic retinopathy, after receiving a single Bevacizumab injection, 16 patients (29.6%) did not show any improvement while 22 (40.7%) patients show an improvement that was less than 50 microns of reduction in macular thickness, 5 patients (9.2%) showed an improvement that was greater than 50 microns in reduction of macular thickness and the remaining 11 patients (20.3%) had a reduction of macular thickness that was greater than 100 microns. Other studies on Bevacizumab injection showed variability in reduction of macular oedema ranging from 36 μm to 110 μm. The FDA approved drug for treatment of DR, ranibizumab also showed mean reduction in central macular thickness (CMT) in range of 118 μm to 196 μm. (Table 6)

TABLE 6
Changes in macular thickness (μm)
after administration of NTGX-10

| | Base- | Reduction | Mean number | Percentage |
|---|---|---|---|---|
| | line | in Macular | of Intravitreal | of Patients |
| | macular | oedema at the | injections in | with CMT |
| Clinical | thick- | end of 12 | span of 12 | less than 275 |
| study | ness | month therapy | months | after 1 year. |
| RESOLVE | 426.6 μm | 118.7 μm | 7 | 49.1% |
| Study | | | Injections | |
| RESOLVE | 455.5 ± | 194.2 μm | 10.2 | NA |
| Study | 114.2 μm | | Injections | |

For assessing the effect of NTGX-10 in patients with Macular, a subgroup analysis was performed in only those eyes of patients which had baseline macular oedema i.e. central macular thickness >300 μm (n=30). After administration of NTGX for 180 days in these patients, statistically significant reduction in central macular thickness was seen. (Table 7)

TABLE 7
Changes in central macular thickness (μm) after administration
of NTGX-10 in patients with baseline macular oedema.

| | Baseline | Day 180 | | |
|---|---|---|---|---|
| Study | (Mean ± SD) | (Mean ± SD) | Difference | |
| variable | N = 30 | N = 30 | (Mean ± SD) | P Value |
| CRT (μm) | 409.77 ± 122.6 | 361.76 ± 127.14 | 48.01 ± 108.5 | 0.02 |

Various clinical trials conducted globally have demonstrated that patients with DR/DME requires frequent intravitreal injections up to 10 injections in one year.

TABLE 8
Number of intravitreal anti VEGF therapy required
by patients in various clinical trials.

| | | | Average |
|---|---|---|---|
| | | | number of |
| | | | injections |
| | | | required in |
| Sr. | Trial | | 12 months |
| No | (Duration) | Patient characteristics | period |
| 1 | ANCHOR | N = 423 Age .50 years Predominantly | 10.7 |
| | (2 years) | classic CNV | |
| 2 | PIER (2 | N = 184 Age .50 years All subfoveal CNV | 6 |
| | years) | of all subtypes | |
| 3 | PrONTO | N = 40 Age .50 years Subfoveal CNV | 5.6 |
| | (2 years) | CRT > 300 μm BCVA 20/40-20/400 | |
| 4 | SUSTAIN | N = 531 Age .50 years; Active subfoveal | 5.3 |
| | (1 year) | AMD CNV; CNV 50% total lesion area; | |
| | | Total lesion size, 12 DA; VA ETDRS 73- | |
| | | 24 (Snellen 20/40-20/320) | |
| 5 | SUSTAIN | N = 4300 Age .50 years Subfoveal AMD | 4.9 |
| | (1 Year) | CNV | |
| 6 | CATT (2 | N = 1107 Treatment naive; VA 20/25- | 5.8 |
| | years) | 20/320; subfoveal AMD CNV | |
| 7 | HORIZON | N = 600 extension study of patients from | 4.2 |
| | (4 years) | MARINA, ANCHOR, FOCUS trials | |

In NTGX-10 clinical trial, only one patient required intravitreal therapy during the study period of 180 days. Intake of NTGX-10 has shown to significantly improve the visual Acuity and also showed to prevent worsening of Macular oedema. Besides this, it also showed to significantly reduce the central retinal thickness. Because of these therapeutic effects, NTGX-10 significantly reduces the requirement of Intravitreal therapy in patients of Diabetic retinopathy. This provides an important positioning to the formulation considering the limitations of intravitreal Anti-VEGF and anti-inflammatory therapy which cannot be administered repeatedly. Findings on Fundus Fluroscence angiography. FF A was done to determine the vascular abnormalities in the eye leading to hemorrhages. The grading was done for each eye according to the ETDRS (Early Treatment Diabetic Retinopathy Study) severity scale for Diabetic retinopathy. Score was given according to pathological characteristics present at various locations in the eye and also a cumulative score was given to each eye. The cumulative score remained constant throughout the treatment duration. Thus no worsening of the grade on the ETD RS scale was observed for all the eyes (N=57) over the treatment period of 6 months indicating NTGX-10 effectively prevents progression of Diabetic Retinopathy. New hemorrhages which may require administration of intravitrial injections were not observed.

Microaneurisms, hemorrhages, neovascularization other ophthalmic complications were evaluated with Fundus Fluorescent Angiography (FFA), Fundoscopy Analysis (FA). Diabetic retinopathy is a progressive disease. Retinal microaneurysms (MAs) are defined as the small swelling of tiny blood vessels, which mainly locate in the inner nuclear layer and deep capillary layer. The number and turnover of retinal MAs are considered as the indicators to assess the presence, severity, and progression risk of retinopathy. Proliferative diabetic retinopathy (PDR) is a major cause of visual loss in diabetic patients and is characterized by neovascularization (NV) that occurs at the vitreoretinal interface and in the vitreous. Neovascularization is clinically characterized by fine loops or networks of vessels lying on the surface of the retina and extending into the vitreous cavity. All of these result in progressive loss of vision, if unchecked. Hence it is important to prevent progression of disease.

In the present study average microaneurysms (Baseline 49.24±46.8, Day 180 43.46±42.2) Neovascularisation of Disc (Baseline 0.43±1.0, Day 180 0.44±1.2), Neovascularization Elsewhere (Baseline 0.34±1.1, Day 180 0.60±1.5), Blot haemorrhages (Baseline 12.09±21.6, Day 180 11.04±19.4) and Dot haemorrhages (Baseline 2.59±7.2, Day 180 1.75±5.3) were stable throughout the study duration indicating slowing of disease progression. ETDRS scale is the disease severity scale for diabetic retinopathy. Worsening of 2 steps or more on ETDRS scale is considered as disease progression. ETDRS grading was done in the present study showed that there was no worsening on the ETDRS scale. This highlights the potential of the study medication to hinder the disease progression.

All the above improvement seen in trial patients were without any significant change in Glycemic control or lipid profile. This rule out the role of above-mentioned confounding variables in the therapeutic outcomes.

### 4.1.5: Study on Secondary Efficacy Variables

The secondary efficacy variables were changes in lipid profile and Glycosylated haemoglobin. Intake of NTGX-10 does not bring any significant changes in HDL, triglycerides, total cholesterol as well as glycemic control. However, a statistically significant change was seen in LDL cholesterol which is of a small magnitude and is not clinically significant. No change was made in any of the concomitant lipid lowering or antidiabetic medication of any study participants.

TABLE 9
Lipid profile and Hb A1C at baseline and postinterventional.

| | | | | | Significance of | |
|---|---|---|---|---|---|---|
| | Day 1 | | Day 180 | | difference in | |
| | (Mean ± SD) | | (Mean ± SD) | | mean P value | |
| HDL | 39.71 | (±9.2) | 39.5 | (±9.5) | 0.7 | (NS) |
| LDL | 122 | (±40.5) | 107 | (±43.7) | 0.01 | |
| Total | 196.24 | (±53.1) | 186.43 | (±58.8) | 0.06 | (NS) |
| cholesterol | | | | | | |
| Triglycerides | 171.1 | (±58.8) | 180.29 | (±141.5) | 0.7 | (NS) |
| Hb A1C (%) | 8.07 | (±2.0) | 8.3 | (±1.6) | 0.2 | (NS) |

NS: Non significant Safety

NTGX-10 was well tolerated in all the patients and none of the patients showed any treatment emergent adverse events. Also, the haematological and biochemical analysis conducted on study participants does not indicate any toxicity in any of the parameters after intake of study medications for 180 days.

NTGX-10 also showed excellent compliance in all the patients.

TABLE 10
Serum Biochemistry and hematology at baseline and day 180.

| | | | | | Significance of | |
|---|---|---|---|---|---|---|
| | Day 1 | | Day 180 | | difference in | |
| | (Mean ± SD) | | (Mean ± SD) | | mean P value | |
| Serum Creatinine | 1.22 | (±0.4) | 1.36 | (±0.7) | 0.16 | (NS) |
| (mg/dl) | | | | | | |
| Total Bilirubin | 0.71 | (±0.2) | 0.73 | (±0.3) | 0.6 | (NS) |
| (mg/dl) | | | | | | |
| AST (U/L) | 20.03 | (±3.5) | 22.4 | (±8.4) | 0.06 | (NS) |
| ALT (U/L) | 21.75 | (±6.5) | 23.45 | (±15.5) | 0.7 | (NS) |
| Hb (gm/dl) | 13.37 | (±1.5) | 13.15 | (±2) | 0.12 | (NS) |
| WBC (cells/ | 7782.59 | (±1772) | 7086.36 | (±1600) | <0.01 | |
| cumm) | | | | | | |
| Platelets | 2.08 | (±0.5) | 2.01 | (±0.6) | 0.3 | (NS) |
| (Lakhs/cumm) | | | | | | |

### Safety Conclusions

A total of 29 TEAEs were reported in 14 (35.0%) patient over the course of the study. No serious adverse event reported during the study.

Out of 29 TEAEs, 10 AEs reported by 8 patients were mild, 18 AEs reported by 9 patients were moderate and 1 AE reported by 1 patient was severe severity.

Out of 29 TEAEs, 1 AE reported by 1 patient was definitely related, 26 AEs reported by 13 patients were not related and 2 AEs reported by 2 patients were unlikely related to study drug.

The commonly (≥5%) reported adverse events were Blood Pressure Increased (5%), Blood Urea Increased (5%), Platelet Count Decreased (5%), Protein Urine Present (5%), Hyperkalaemia (5%), Blood Creatinine Increased (7.5%) and Proteinuria (7.5%).

In summary, based on the analysis of adverse events, it is demonstrated that NTGX-10 is safe and well tolerated.

### 4.2 Case Studies

### 4.2.1: Case Study 1

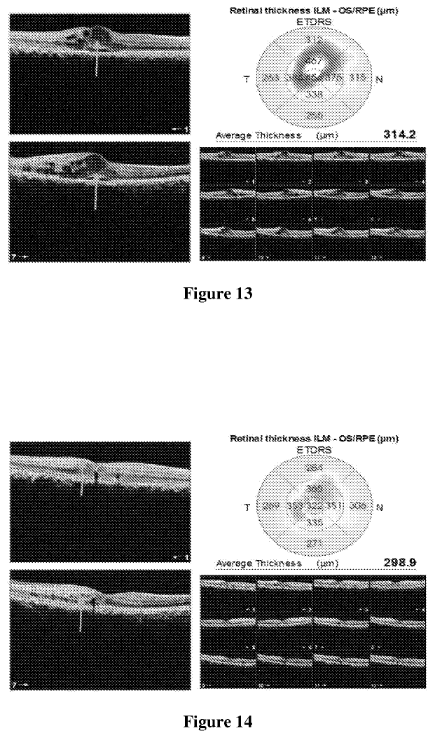

A male patient of age 54 years having diabetes since 2007 came to Shalakya OPD on Jul. 6, 2019 for the complaint of blurred vision. Local eye examination was carried out and visual acuity was measured. Fundoscopy and ophthalmoscopy revealed presence of Diabetic Macular Edema (DME) characteristic feature of Diabetic Retinopathy. OCT was performed for assessment of DME. The Baseline central macular thickness (CMT) for Right Eye was 456 μm and that of ETDRS field 2 was 467 μm. OCT image showed presence of DME as observed in FIG. 13. Treatment was initiated with 1 capsule of NTGX-10 twice a day. Patient was instructed to mix the capsule contents in unequal proportion of honey and ghee and consume at least half an hour before food. The treatment was continued for 6 months and OCT was performed every 2 months. After 6 months of treatment considerable reduction in macular edema was observed and macular thickness was restored to normal range. The central macular thickness of left eye was found to be 322 μm (reduced by 134 μm) and that of ETDRS field 2 was found to be 365 μm (reduced by 102 μm). OCT image showed resolution of DME as observed in FIG. 14. The results are presented at Table 11.

TABLE 11
Maccular Thickness of ETDRS fields of Left Eye.

Maccular Thickness (μm)

| Field No. | Baseline | Day 180 |
|---|---|---|
| 1 | 456 | 322 |
| 2 | 467 | 365 |
| 3 | 375 | 351 |
| 4 | 338 | 335 |
| 5 | 384 | 353 |
| 6 | 312 | 284 |
| 7 | 315 | 306 |
| 8 | 255 | 271 |
| 9 | 263 | 269 |

### 4.2.2. Case Study 2

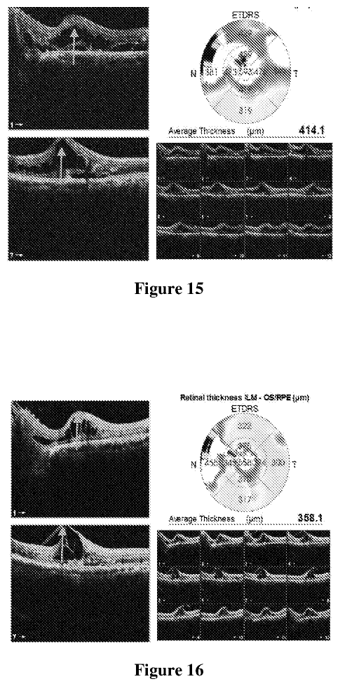

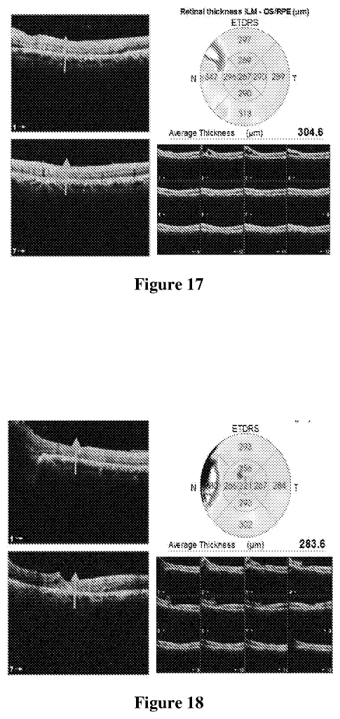

A male patient of age 54 years having diabetes since 2014 came to Shalakya OPD on Jul. 6, 2019 for the complaint of blurred vision. Local eye examination was carried out and visual acuity (1.8 Log MAR Units) was measured. Fundoscopy and ophthalmoscopy revealed presence of Diabetic Macular Edema (DME) characteristic feature of Diabetic Retinopathy. OCT was performed for assessment of DME. The Baseline central macular thickness for Left Eye was 593 μm. OCT image showed presence of DME as observed in FIG. 15. Treatment was initiated with 1 capsule of NTGX-10 twice a day. Patient was instructed to mix the capsule contents in unequal proportion of honey and ghee and consume at least half an hour before food. The treatment was continued for 6 months and OCT was performed every 2 months. The macular thickness was reduced after 2 months of treatment (CMT left eye 558 μm) and was consistently reduced after 4 months of treatment (CMT left eye 267 μm) and after 6 months of treatment (CMT left eye 221 μm). The overall reduction in macular thickness was 372 μm. OCT image showed reduction in DME after 2 months of treatment (FIG. 16) and resolution of DME after 4 months of treatment (FIG. 17) and after 6 months of treatment (FIG. 18). Visual Acuity was found to be 0.8 Log MAR Units after 6 months of treatment. Results are presented at Table 12.

TABLE 12
Maccular Thickness of ETDRS fields of Left Eye.

Maccular Thickness (μm)

| Field No. | Baseline | Day 60 | Day 120 | Day 180 |
|---|---|---|---|---|
| 1 | 593 | 558 | 267 | 221 |
| 2 | 459 | 375 | 269 | 256 |
| 3 | 473 | 345 | 296 | 266 |
| 4 | 419 | 370 | 290 | 293 |
| 5 | 476 | 374 | 290 | 267 |
| 6 | 429 | 322 | 297 | 293 |
| 7 | 381 | 455 | 347 | 280 |
| 8 | 319 | 317 | 313 | 302 |
| 9 | 449 | 300 | 289 | 284 |

### 4.2.3. Case Study 3

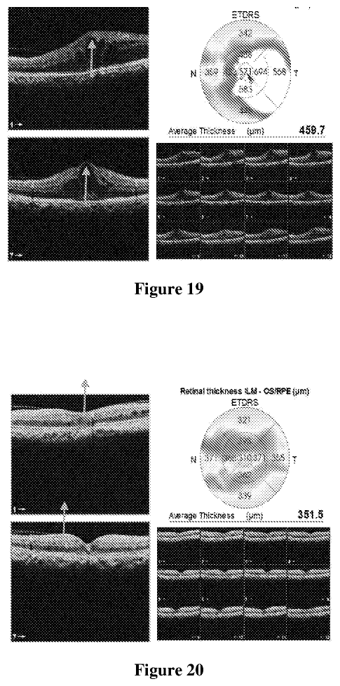

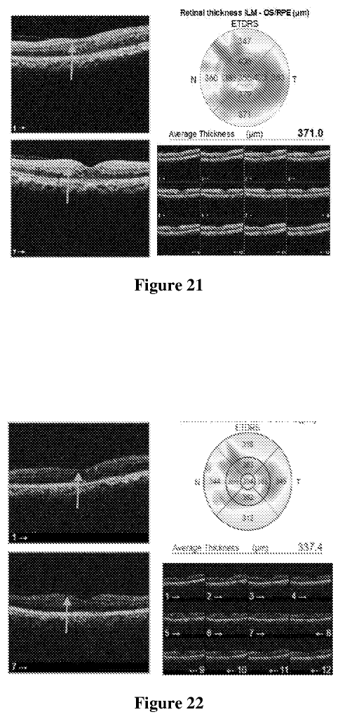

A male patient of age 62 years having diabetes since 2006 came to Shalakya OPD on Jul. 7, 2019 for the complaint of blurred vision. Local eye examination was carried out and visual acuity (1 Log MAR Unit) was measured. Fundoscopy and ophthalmoscopy revealed presence of Diabetic Macular Edema (DME) characteristic feature of Diabetic Retinopathy. OCT was performed for assessment of DME. The Baseline central macular thickness (CMT) for Left Eye was 571 μm. OCT image showed presence of DME as observed in FIG. 19. Treatment was initiated with 1 capsule of NTGX-10 twice a day. Patient was instructed to mix the capsule contents in unequal proportion of honey and ghee and consume at least half an hour before food. The treatment was continued for 6 months and OCT was performed every 2 months. The macular thickness was reduced after 2 months of treatment (CMT left eye 355 μm) and was consistently reduced after 4 months of treatment (CMT left eye 310 μm) and after 6 months of treatment (CMT left eye 304 μm). The overall reduction in macular thickness was 267 μm. OCT image showed reduction in DME after 2 months of treatment (FIG. 20) and resolution of DME after 4 months of treatment (FIG. 21) and after 6 months of treatment (FIG. 22). Visual Acuity was found to be 0.5 Log MAR Units after 6 months of treatment. Results are presented at Table 13.

TABLE 13
Maccular Thickness of ETDRS fields of Left Eye.

Maccular Thickness (μm)

| Field No. | Baseline | Day 60 | Day 120 | Day 180 |
|---|---|---|---|---|
| 1 | 571 | 355 | 310 | 304 |
| 2 | 488 | 406 | 393 | 383 |
| 3 | 433 | 385 | 368 | 356 |
| 4 | 583 | 379 | 362 | 362 |
| 5 | 694 | 400 | 371 | 365 |
| 6 | 342 | 347 | 321 | 318 |
| 7 | 359 | 369 | 371 | 344 |
| 8 | 445 | 371 | 339 | 312 |
| 9 | 568 | 383 | 351 | 345 |

### 4.2.4. Case Study 4

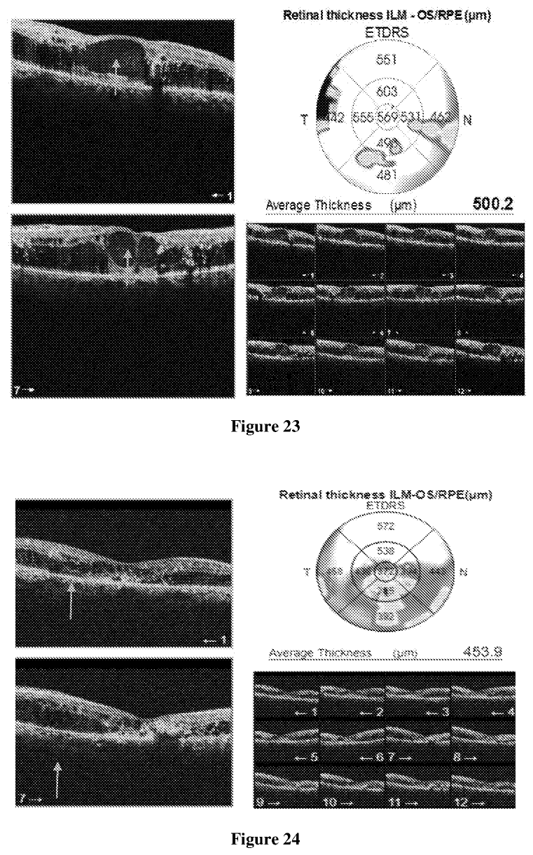

A female patient of age 59 years having diabetes since 2007 came to Shalakya OPD on Apr. 10, 2019 for the complaint of blurred vision. Local eye examination was carried out and visual acuity (0.8 Log MAR Units) was measured. Fundoscopy and ophthalmoscopy revealed presence of Diabetic Macular Edema (DME) characteristic feature of Diabetic Retinopathy. OCT was performed for assessment of DME. The Baseline central macular thickness (CMT) for Right Eye was 569 μm. OCT image showed presence of DME (FIG. 23). Treatment was initiated with 1 capsule of NTGX-10 twice a day. Patient was instructed to mix the capsule contents in unequal proportion of honey and ghee and consume at least half an hour before food. The treatment was continued for 6 months and OCT was performed every 2 months. The macular thickness was reduced after 6 months of treatment (CMT Right eye 372 μm). The overall reduction in macular thickness was 197 μm. OCT image showed reduction in DME after 4 months of treatment (FIG. 24). Visual Acuity was found to be 0.3 Log MAR Units after 6 months of treatment. Results are presented at Table 14.

TABLE 14
Maccular Thickness of ETDRS fields of Right Eye.

Maccular Thickness (μm)

| Field No. | Baseline | Day 180 |
|---|---|---|
| 1 | 569 | 372 |
| 2 | 603 | 538 |
| 3 | 531 | 394 |
| 4 | 498 | 285 |
| 5 | 555 | 439 |
| 6 | 551 | 572 |
| 7 | 462 | 443 |
| 8 | 481 | 398 |
| 9 | 442 | 468 |

### 4.3. Angiogenic Screening of NTGX-10, Bioprocessed Gold and Bioprocessed Zinc Employing in Ovo-In Vivo Chick Embryo Yolk Sac Membrane (YSM) Model

### 4.3.1. Introduction

Angiogenesis, the process by which new blood vessels are formed from the pre-existing ones, is eminent for the growth and development of an organism. In an adult organism physiological angiogenesis occurs cyclically in female reproductive system and in wound healing. Impaired angiogenesis is seen in disease conditions such as arthritis, diabetic retinopathy, macular degeneration and cancerous tumors (Molecular Vision 2016; 22:436-445; Nutrients 2016; 8 (4): 200, BMC Cancer. 2015; 15 (1): 1-7). On the other hand, anti-angiogenesis—the prevention of the growth of new blood vessels is of key importance in treatment of cancers (Clinical Therapeutics. 2006; 28 (11): 1779-1802); A Cancer Journal for Clinicians. 2010; 60 (4): 222-243; Nature Reviews Clinical Oncology. 2009; 6 (8): 465-477; Cancer Treatment Reviews 2011; 37 (1): 63-74). Under normal physiological conditions equilibrium between the pro- and anti-angiogenic factors maintains a stable state microcirculation so that blood vessels are formed only when and where they are needed (J Biol Chem. 1992; 267:10931-10934). Disturbed equilibrium with excess angiogenic factors causes increase in the rate of capillary endothelial cell migration and proliferation resulting into excess vascularization (Journal of cellular biochemistry. 1985; 29:275-28). Oppositely excess anti-angiogenic factors cause reduction in the number of existing blood vessels by blocking various steps in the process of blood vessels formation. Drug development for the treatment of diseases like ischemia of heart and brain as well as cancers and tumours need appropriate preclinical screening assays using animal models. Various model systems have been reported in the literature. In the present study angiogenic screening of NTGX-10, Bioprocessed Gold and Bioprocessed Zinc has been carried out by employing in ovo-in vivo chick embryo Yolk Sac Membrane (YSM) model system (Journal of cellular and molecular medicine. 2006: 10(3):588-612; British journal of experimental pathology 1987: 68(6):755); Mechanisms of development 2076; 141:70-7; (CAM assay. 2073; 3(8): 1-7) was provided by Applicant as a positive control. Anti-angiogenesis starves the tumor from all its requirements resulting into its death. Hence, it is the major treatment option in clinical oncology. Quality of blood vessel plays an important role in the cure of cancer through anti-angiogenic pathways.

### 4.3.2 Objectives

To identify the angiogenic/anti-angiogenic potential of NTGX-10 employing in ovo-in vivo chick embryo Yolk Sac Membrane (YSM) model system.

### 4.3.3. Experimental Procedure

Test Item Item Code: NTGX-10, Bioprocessed Gold, Bioprocessed Zinc Batch No: NTGX-10-APPLICANT/SEP-17/SUB-01; Bioprocessed Gold-RB/BCNS-Au/05/18/001; Bioprocessed Zinc-RB/BCNS-Zn/05/18/001 Supplied by: Rasayani Biologics Pvt Ltd. Physical Appearance: NTGX-10: Greenish powder, Bioprocessed Gold: Greyish and Bioprocessed Zinc: Greyish Storage: Room temperature (27° C.).

### 4.3.3. Test System

In ovo-in vivo chick embryo Yolk Sac Membrane (YSM) model employing freshly fertilized eggs incubated for 72 h.

### 4.3.4 Justification

YSM is a well vascularized membrane present from 3rd to 6th day of incubation during development of chick embryo. During this duration the vascularized area of the membrane is used as a tissue to study angiogenic/anti-angiogenic responses of biological or synthetic agents Rosenbruch M. Hoist A. The chick embryo yolk-sac blood vessel system as an experimental model for irritation and inflammation. (Toxicology in vitro. 1990; 4:327-31, Murray J C. 2001 Angiogenesis Protocols. Methods in molecular medicine. New Jersey: Humana Press).

### 4.3.5 Dose Preparation and Administration

To prepare a stock solution (A) of 1 mg/ml concentration, 1 mg each of NTGX-10, Bioprocessed Gold and Bioprocessed Zinc were weighed and each mixed in 1 ml of 0.9% sterile saline (isotonic). 81 ml solution of NTGX-10, Bioprocessed Gold and Bioprocessed Zinc with concentration of 100 μg/ml (B) was prepared from solution A (100 μl solution A+900 μl saline). 200 μl solution each of 10 μg/ml (20 μl stock solution B+180 μl 0.9% sterile saline) and 40 μg/ml (80 μl stock solution B+120 μl 0.9% sterile saline) of the selected concentrations of NTGX-10, Bioprocessed Gold and Bioprocessed Zinc were prepared. 40 μl of each dose was released directly on vascularized area of YSM with the help of micropipette.

### 4.3.6 Avastin Preparation

Stock solution of Avastin (A) of the concentration of 25 mg/ml was supplied by APPLICANT. Ten microliter working stock (B) of Avastin of the concentration of 2.5 mg/ml was prepared (1 μl of stock B+9 μl of 0.9% saline). One ml of Avastin (15 μg/ml) was prepared from the working stock B (6 μl B+994 μl 0.9% saline). Dose of 40 μl was released directly on vascularized area of YSM with the help of micropipette.

### 4.3.7 Protocol

Freshly fertilized eggs of White Leghorn breed of fowls weighing 58-60 g each procured from Venkateshwara Hatcheries Pvt. Ltd., Khadakwasala, Pune, Maharashtra, were washed with tap water to remove dirt and soiling, if any, and allowed to air dry. Eggs were then incubated at 37.5° C. with a relative humidity of 70-80% for 72 hr. Stage of development was confirmed by candling the eggs with 40 watts bulb. Embryos showing synchronized development were selected for the experiments. Eggs with live embryos at the HH stage 18-24 (Hamburger V, Hamilton HL. A series of normal stages in the development of chick embryos. Journal of Morphology. 1951; 88:49-92 having well vascularized YSM were surface sterilized with 70% alcohol in the laminar flow hood. 1-2 ml thin albumen was sucked out through an aperture drilled in the egg shell. The aperture was then sealed with dorapore tape. YSM was exposed by cutting the shell at the blunt end. 10, 40 and 100 μg/ml concentration of drugs was chosen for the assay and dilutions were made in 0.9% sterile saline. 40 μl of drug preparation was directly released over the vasculature using a micropipette and egg shells were closed using dorapore tape. Eggs were again incubated for 24 hours at 37.5° C. and 70-80% humidity. For master control YSM was not treated with any agent, for vehicle control YSM is treated with 0.9% saline. Eggs were opened by removing the dorapore tape, some more thin albumen is removed with the micropipette, egg shell is cut to expose the YSM vasculature and images were taken and images of control and experimental (YSM) were captured for comparative studies and quantitation using Olympus D40 SLR camera. Images of YSM were cropped and resized to 992×992 pixels. A black square of size 300×300 pixels were placed in the areas where the effect of test substance was seen. Number of primary, secondary, tertiary and quaternary blood vessels were counted manually using Image J software. Avastin (Yildiz et. al., 2013) was provided by RBPL as a positive control.

NTGX-10, Bioprocessed Gold and Bioprocessed Zinc were screened to study its effects on development of quaternary blood vessels employing in ovo-in vivo chick embryo YSM model. Three different concentrations viz. 10 μg/ml, 40 μg/ml and 100 μg/ml were applied and observations were recorded after 24 hrs of incubation. It was observed that at all the concentrations tested i.e. 10 μg/ml, 40 μg/ml and 100 μg/ml NTGX-10 exhibits significant decrease in the number of quaternary blood vessels of YSM in comparison with Master Control (MC) and Vehicle Control (VC i.e. 0.9% saline), Bioprocessed Gold and, Bioprocessed Zinc. The number of quaternary blood vessels of YSMs treated with all the three concentrations was observed to be comparable to that of the positive control viz. Avastin (15 μg/ml). However, amongst the concentrations tested, NTGX-10 at 40 μg/ml, Bioprocessed Gold at 100 μg/ml as well as 40 μg/ml, and Bioprocessed Zinc at 100 μg/ml concentration, exhibits maximum anti-angiogenic activity. This suggests that NTGX-10, Bioprocessed Gold and Bioprocessed Zinc are a product with antiangiogenic potential at the concentrations of 10 μg/ml, 40 μg/ml and 100 μg/ml.

### Results.

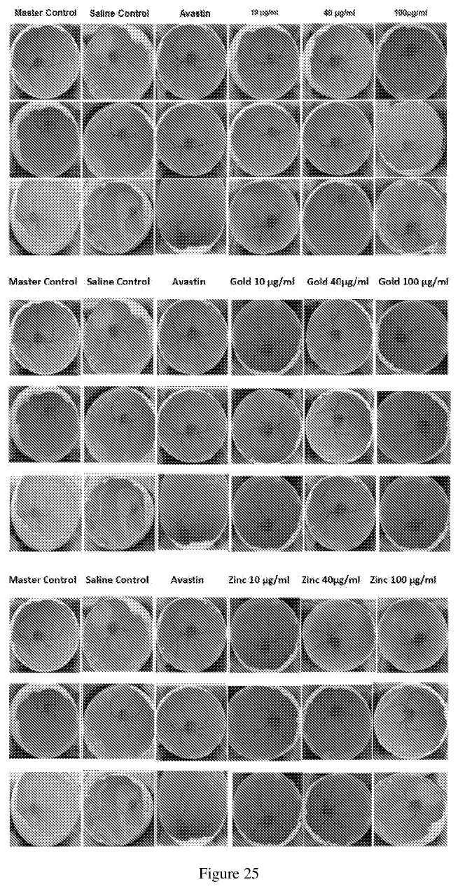

Photographs of the YSM model captured 24 h after the treatment with selected doses of NTGX10, Bioprocessed Gold and Bioprocessed Zinc (FIG. 25).

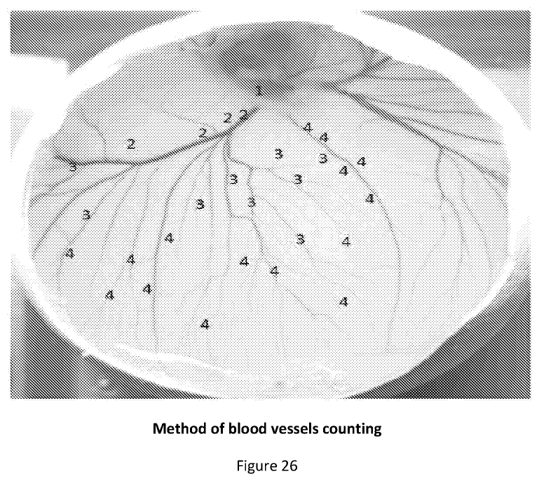

Blood vessels arising next to the heart of embryo are counted as—primary vessels. Branches of primary blood vessels are the secondary vessels, branches of secondary are the tertiary and branches of tertiary blood vessels are the quaternary blood vessels. Angiogenesis is the process of formation of new blood vessels from the pre-existing vessels. Hence quantitation of angiogenesis is carried out with reference to the increase or decrease in the number of quaternary blood vessels (FIG. 26).

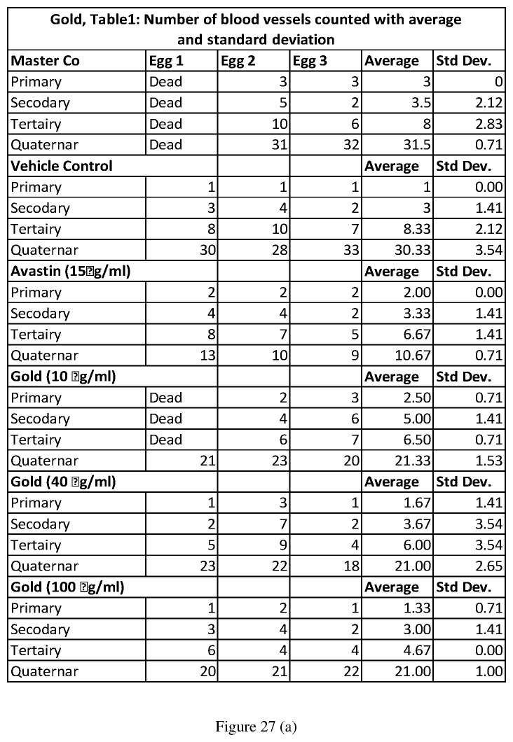

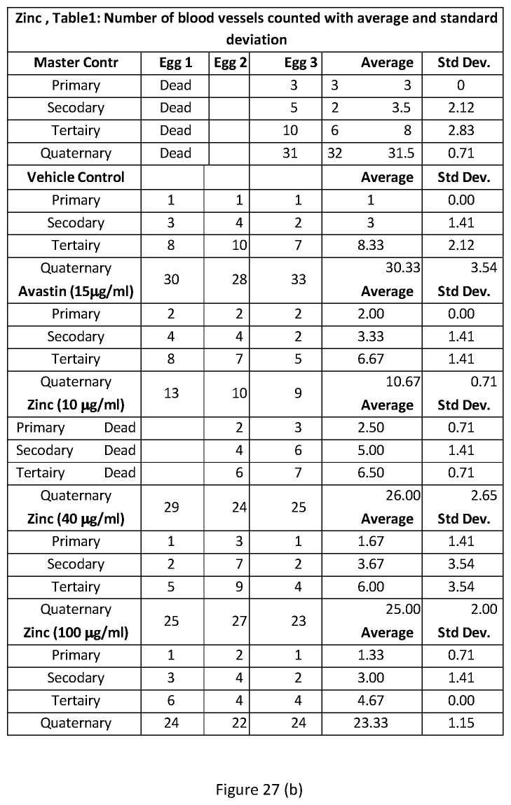

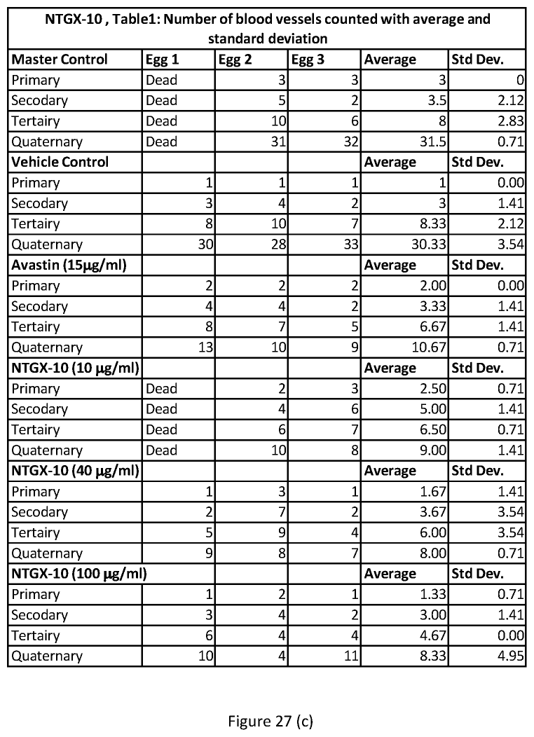

Primary, secondary, tertiary and quaternary blood vessels were counted in individual YSM 24h after the treatment with selected doses of NTGX-10, Bioprocessed Gold and Bioprocessed Zinc. Results of the counting of blood vessels in individual YSM in the form of an excel sheet are expressed. Blood vessels arising next to the heart of embryo are counted as—primary vessels. Branches of primary blood vessels are the secondary vessels, branches of secondary are the tertiary and branches of tertiary blood vessels are the quaternary blood vessels were counted in individual YSM 24h after the treatment with selected doses of NTGX-10, Bioprocessed Gold and Bioprocessed Zinc. Results of the counting of blood vessels in individual YSM in the form of an excel sheet are expressed in FIG. 27.

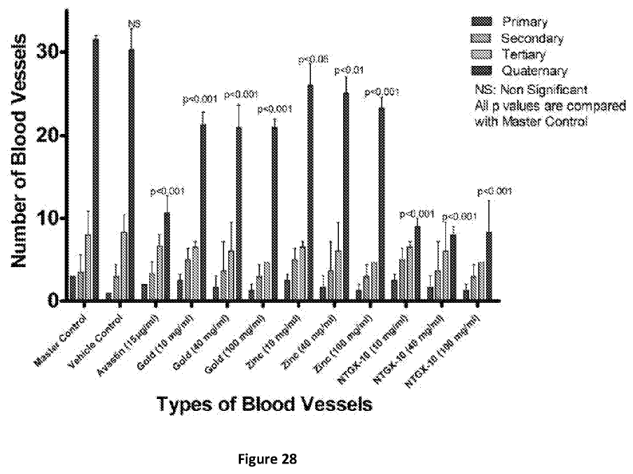

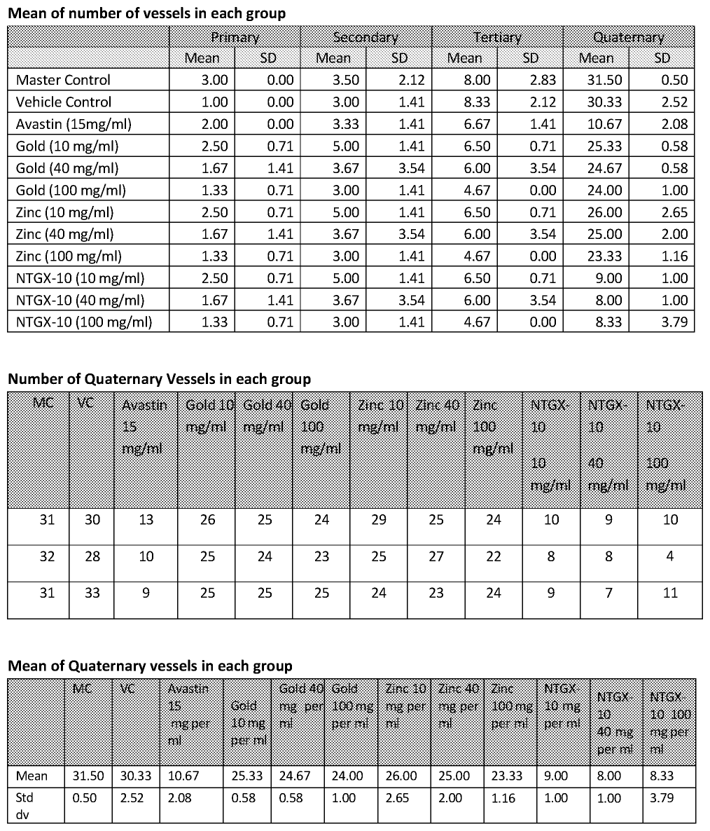

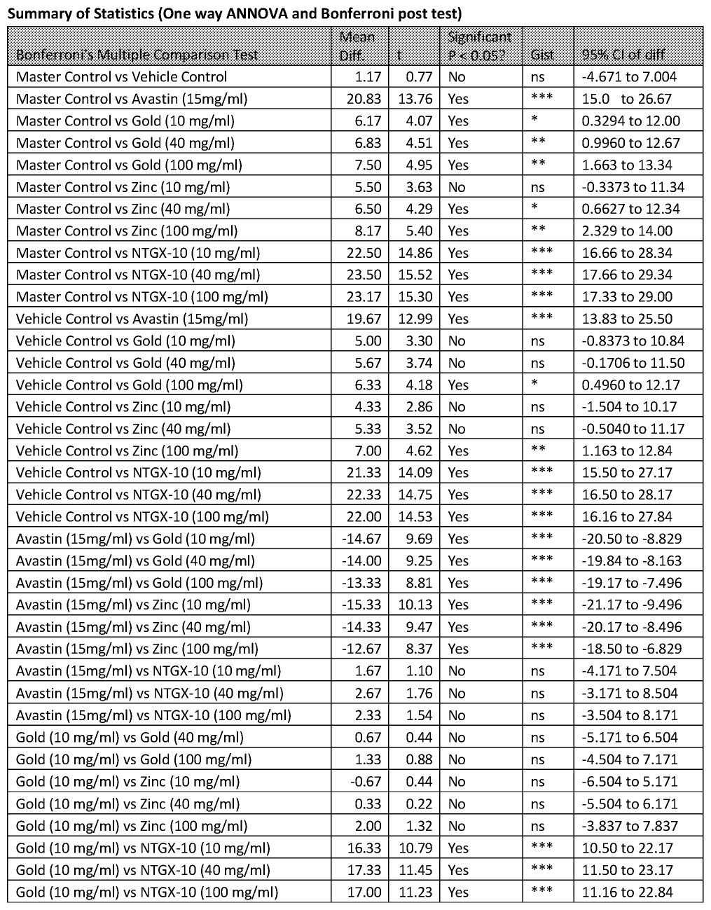

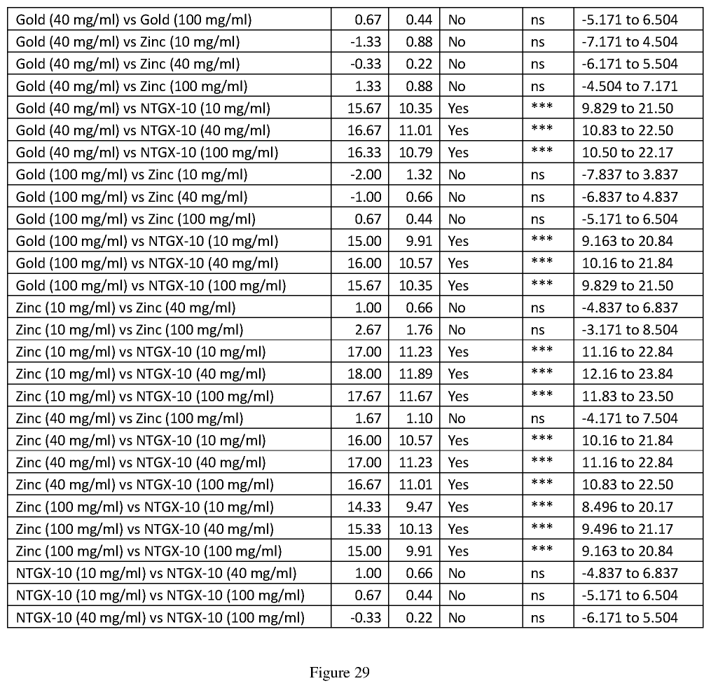

Graphical representation of the results carried out using sigma plot software is attached as FIG. 28. Statistical analysis of results of the counting of number of each types of blood vessels in individual YSM was carried out using One Way Analysis of Variance using sigma stat software. System 12 generated sheet of results is attached as FIG. 29.

Anti-angiogenic potential was shown by all the studied drugs at their respective concentrations. Among studied all the drugs NTGX-10, comprising a combination of bio-gold and bio-zinc showed the higher anti-angiogenic potential.

1. A combination of bio-gold and bio-zinc for oral administration for its utility in alleviating micro & macro vascular complications.

2. The combination as claimed in claim 1, wherein the bio-gold is highly crystalline face-centered cubic (FCC) phase of gold.

3. The combination as claimed in claim 1, wherein the bio-gold is present as a nano particulate system with bio-gold as spherical or distorted spherical particles in the core of the system.

4. The combination as claimed in claim 1, wherein the nanoparticulate spherical or distorted spherical bio-gold is surrounded by amorphous inorganic matrix, of polydispersed irregular shaped or regular shaped particles selected from group consisting of triangular, rectangular, rod-like, and spherical particles and their mixtures thereof composed of amorphous metal or amorphous metal oxides or mixtures thereof in the particle-size range of 0.5 microns to 10 microns.

5. The combination as claimed in claim 1, wherein the bio-gold comprises peaks related to elemental gold (Au⁰) and oxidated gold Au¹⁺ in the ratio of 2:3.

6. The combination as claimed in claim 1, wherein the bio-zinc is present as hexagonal phase of Zinc Oxide (ZnO) and without any free Zinc.

7. The combination as claimed in claim 1, wherein the bio-zinc is present as agglomerated, heterogenous polydispersed irregular shaped or regular shaped particles selected from group consisting of triangular, rectangular, rod-like, and spherical particles and their mixtures thereof composed of amorphous metal or amorphous metal oxides or mixtures thereof in the particle-size range of 10 nM to 1000 nM.

8. The combination as claimed in claim 1, wherein the bio-zinc comprises additional elements selected from the group consisting of Zn, O, Mg, Si, C, K, Ca, or combinations thereof.

9. The combination as claimed in claim 1, wherein the bio-zinc comprises a coating of MgO, SiO₂, CaO, are forming the amorphous matrix around submicron scale particles and nanoparticles of ZnO.

10. The combination as claimed in claim 1, wherein bio-gold is in the range 2 to 4% (w/w);
bio-zinc in the range of 3% to 5% (w/w) and in the ratio of 1:1 to 1:4.

11. A process for preparation of bio-gold as claimed in claim 1, comprising the steps of:
(i) obtaining metallic gold of purity 99.99% in the form of foils/powder/flakes,
(ii) heating gold of step (i) until the gold is red hot,
(iii) quenching the red-hot gold of step (ii) in the aqueous extract of rhizome of Curcuma longa,
(iv) carrying out steps (ii) and (iii) in cycles for 10 to 100 times, preferably 15 to 50 times, more preferably for 20 to 40 times to obtain a brittle gold,
(v) triturating the brittle gold of step (iv) with the aqueous extract of rhizome of Curcuma longa in the ratio of 1:1 to 1:2 for a period of 6-8 hrs to obtain a powder,
(vi) placing the powder in a covered earthen vessel and heating the mixture for 100° C. to 1000° C., preferably 350° C. to 850° C., more preferably to 450° C.-750° C. for a period of 20-45 min. and then allowed to cool gradually to room temperature,
(vii) carrying out steps (v) and (vi) in cycles for 10 to 500 times, preferably 50 to 250 times, more preferably for 80 to 120 times to obtain a finely divided pinkish colored final bio-gold in the particle size ranging from submicron to nano scale.

12. A process for preparation of bio-zinc as claimed in claim 1, comprising the steps of:
i. obtaining metallic Zinc of purity 99.99% in the form of foils/powder/flakes,
ii. heating of zinc of step (i) till it completely liquifies,
iii. quenching the liquefied zinc of step (ii) in the aqueous extract of the pulp of the fruits Phyllathus embellica after removing the seeds,
iv. carrying out steps (ii) and (iii) in cycles for 10 to 100 times, preferably 15 to 75 times, more preferably for 20 to 40 times to obtain a brittle zinc,
v. triturating the brittle zinc of step (iv) with the aqueous extract of the pulp of the fruits Phyllathus embellica in the ratio of I:I to I:2 for a period of 6-8 hrs to obtain a powder,
vi. placing the powder in a covered earthen vessel and heating the mixture for 100° C. to 1000° C., preferably 250° C. to 750° C., more preferably to 350° C.-600° C. for a period of 20-45 min. and then allowed to cool gradually to room temperature,
vii. carrying out steps (v) and (vi) in cycles for 10 to 100 times, preferably 20 to 80 times, more preferably for 30 to 60 times to obtain a finely divided powder of bio zinc of the present invention.

13. A process for obtaining the combination of claim 1, comprising the step of combining bio-gold as claimed in claim 11 and the bio-zinc as claimed in claim 12 in the ratio of 1:1 to 1:4.

14. A composition, containing the combination as claimed in claim 1 along with pharmaceutically acceptable excipients.

15. A composition as claimed in claim 14, wherein the bio-gold in the range 2 to 4% (w/w);
bio-zinc as in the range of 3 to 5% (w/w) and pharmaceutically acceptable excipients in the range of 91 to 95% (w/w).

16. The combination or composition as claimed in claim 1, as an oral dosage form in a dose of 0.02-2.00 mg/Kg.

17. The combination or composition as claimed in claim 1, for its utility in alleviating micro and macrovascular complications.

18. The combination or composition as claimed in claim 1, for its utility in diabetic macular edema (DME), in Diabetic retinopathy.

19. The combination or composition as claimed in claim 1, for its action in mammals independent of pathology.
